(19) United States
(12) Patent Application Publication
(10) Pub. No.: US 2025/0302889 A1
(43) Pub. Date: Oct. 2, 2025

(54) Ingestible Nutritive Compositions With Prebiotics, Probiotics, and Postbiotics

(71) Applicant: First Day Life, Inc., Austin, TX (US)

(72) Inventors: Bridgette Park, Granada Hills, CA (US); Shuyan Li, Austin, TX (US)

(21) Appl. No.: 18/621,753

(22) Filed: Mar. 29, 2024

(51) Int. Cl. A61K 35/742; A61K 35/744; A61K 36/185; A61P 1/00; A61P 1/02; A61P 1/12; A61P 3/06; A61P 17/00; A61P 29/00; A61P 31/04; A61P 31/14; A61P 31/16; A61P 37/04

(52) U.S. Cl. CPC A61K 35/742; A61K 35/744; A61K 36/185; A61P 1/00; A61P 1/02; A61P 1/12; A61P 3/06; A61P 17/00; A61P 29/00; A61P 31/04; A61P 31/14; A61P 31/16; A61P 37/04

(57) ABSTRACT

Compositions including prebiotics, probiotics, and postbiotics for oral administration and methods of using such compositions for improving health of subjects are provided.

### BACKGROUND OF THE INVENTION

The present invention generally relates to nutritional compositions comprising prebiotics, probiotics, and postbiotics for oral administration and methods for using the oral compositions in improving health of subjects (e.g., human subjects).

### BRIEF SUMMARY OF THE INVENTION

In an aspect of the invention, an orally ingestible nutritional composition for improving health is provided. The composition comprises an amount of gold kiwifruit powder, an amount of Bacillus coagulans, and an amount of heat-treated Lactococcus lactis. The gold kiwifruit powder within the composition may include or consist essentially of Livaux®. The Bacillus coagulans within the composition may include or consist essentially of UNIQUE IS-2®. The heat-treated Lactococcus lactis within the composition may include or consist essentially of IMMUSE™.

In some embodiments, components of the inventive composition may be combined at a w/w ratio of between about 2:1 and about 15:1 gold kiwifruit powder: Bacillus coagulans. In some embodiments, components of the inventive composition may be combined at a w/w ratio of between about 5:1 and about 20:1 gold kiwifruit powder: Lactococcus lactis. In some embodiments, components of the inventive composition may be combined at a w/w ratio of between about 1:1 and about 5:1 Bacillus coagulans:Lactococcus lactis.

In some embodiments, components of the inventive composition may be combined at a w/w ratio of between about 5:1 and about 20:1 gold kiwifruit powder: Bacillus coagulans. In some embodiments, components of the inventive composition may be combined at a w/w ratio of between about 3:1 and about 7:1 gold kiwifruit powder: Lactococcus lactis. In some embodiments, components of the inventive composition may be combined at a w/w ratio of between about 1.5:1 and about 3.5:1 Bacillus coagulans: Lactococcus lactis.

In some embodiments, the orally ingestible compositions may take the form of gummies, tablets, capsules, liquids, suspensions, chewables, soft gels, sachets, powders, syrups, liquid suspensions, emulsions, or other solutions. In particular embodiments, the orally ingestible composition is in the form of an individual dosage form. The individual dosage form may take the form of a gummy, a tablet, a capsule, a soft gel, a sachet, or a chew. In particular embodiments, the individual dosage form is a gummy. In further embodiments, an individual oral dosage form may comprise between about 75 mg and 700 mg gold kiwifruit powder; between about 5 mg and 80 mg heat-treated Lactococcus lactis; and between about 5 mg and 200 mg Bacillus coagulans. In particular embodiments, the individual oral dosage form comprises about 125 mg gold kiwifruit powder; about 18 mg heat-treated Lactococcus lactis; and about 25 mg Bacillus coagulans. In particular embodiments, an individual oral dosage form may comprise an amount of gold kiwifruit powder, an amount of heat-treated Lactococcus lactis, and between about 500 million CFUs and 20 billion CFUs Bacillus coagulans. In particular embodiments, the individual oral dosage form comprises an amount of gold kiwifruit powder, an amount of heat-treated Lactococcus lactis, and about 2.5 billion CFUs Bacillus coagulans.

In an aspect, there is a method for administering orally ingestible compositions to a subject in accordance with the disclosure. In some embodiments, the subject is a mammal. In some embodiments, the subject is a human. In some embodiments, the subject is an adult human. In particular embodiments, the subject is an adult human female. In particular embodiments, the subject is an adult human male. In particular embodiments, the subject is an elderly adult human. In still further embodiments, the subject is an elderly adult female. In still further embodiments, the subject is an elderly adult male. In some embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human teenager between the ages of 13 and 19.

Compositions according to embodiments herein may be administered a single time, daily on a continual basis, or multiple times per week depending on subject needs, subject body size, and on safety. Dosing may occur once, or only a weekly or monthly basis. Dosage level and frequency of administration protocols, compositions, as well as potential use of additional therapeutic or nutritive agents in combination can be adjusted during the course of administration. In an embodiment, compositions in accordance with the present inventions can be administered daily to a subject. In a particular embodiment, compositions in accordance with the present inventions can be administered multiple times per day to a subject.

In some embodiments, the subject receives, via oral administration of compositions in accordance with the disclosure herein, between about 75 mg and 2800 mg of gold kiwifruit powder per day.

In some embodiments, the subject receives, via oral administration of compositions in accordance with the disclosure herein, between about 5 mg and 320 mg of heat-treated Lactococcus lactis per day.

In some embodiments, the subject receives, via oral administration of compositions in accordance with the disclosure herein, between about 5 mg and 800 mg of Bacillus coagulans per day. In some embodiments, the subject receives between about 500 million CFUs and 80 billion CFUs Bacillus coagulans per day.

In an embodiment, a method of increasing the amount of Faecalibacterium prausnitzii within a digestive tract of a subject is provided. The method comprises orally administering, to the subject, an orally ingestible composition in accordance with the disclosure herein. In particular embodiments, the subject is a human. In particular embodiments, the subject is a human adult. In further embodiments, the subject is a human child under the age of 18. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to achieve an increase in the amount of Faecalibacterium prausnitzii within the digestive tract of the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In an embodiment, a method of reducing constipation or relieving symptoms of constipation in a subject is provided. The method comprises orally administering, to the subject, an orally ingestible composition in accordance with the disclosure herein. In particular embodiments, the subject is a human. In particular embodiments, the subject is a human adult. In further embodiments, the subject is an adult human female. In particular embodiments, the subject is a human child under the age of 18. In some embodiments, the subject is a human teenager between the ages of 13 and 19.

In some embodiments, the method comprises orally delivering to the subject an effective amount or concentration of one or more of gold kiwifruit powder, heat-treated Lactococcus lactis, and Bacillus coagulans to achieve a reduction in constipation or relief of symptoms of constipation in the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments, the subject's frequency of complete spontaneous bowel movement is increased post-administration of the orally ingestible composition.

In some embodiments, the subject's stools are softened and/or improved in consistency post-administration of the orally ingestible composition as measured by the Bristol stool form scale.

In some embodiments, the subject's pain during defecation is reduced post-administration of the orally ingestible composition as measured by the constipation scoring system (CSS) scale.

In some embodiments, the subject's abdominal pain associated with constipation is reduced post-administration of the orally ingestible composition as measured by the constipation scoring system (CSS) scale.

In an embodiment, a method of reducing bacterial vaginosis symptoms in a human female subject is provided. The method comprises orally administering, to the human female subject, an orally ingestible composition in accordance with the disclosure herein. In particular embodiments, the subject is an adult human female. In particular embodiments, the subject is a female human child under the age of 18. In some embodiments, the subject is a female human teenager between the ages of 13 and 19. In some embodiments, the method comprises orally delivering to the subject an effective amount or concentration of one or more of gold kiwifruit powder, heat-treated Lactococcus lactis, and Bacillus coagulans to reduce bacterial vaginosis symptoms in the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day. In some embodiments, the human female subject is undergoing antibiotic treatment for bacterial vaginosis.

In an embodiment, a method of improving the immune response of a human subject is provided. The method comprises orally administering, to the subject, an orally ingestible composition in accordance with the disclosure herein. In particular embodiments, the subject is a human child under the age of 18. In particular embodiments, the subject is a human adult. In some embodiments, the subject is a human teenager between the ages of 13 and 19. In some embodiments, the method comprises orally delivering to the subject an effective amount or concentration of one or more of gold kiwifruit powder, Lactococcus lactis, and Bacillus coagulans to improve the immune response of the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments, incidence frequency of gastrointestinal (GI) infections and/or symptoms associated therewith is decreased in the human subject post-administration of the orally ingestible composition.

In some embodiments, incidence frequency of upper respiratory tract infections is decreased in the human subject post-administration of the orally ingestible composition.

In some embodiments, the incidence frequency of colds and cold-like illness in the human subject is decreased post-administration of the orally ingestible composition.

In an embodiment, a method of improving oral health and reducing tooth decay in a human subject is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In further embodiments, the human subject is an adult human male. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to improve the oral health of and reduce tooth decay in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments, salivary Streptococcus mutans content is decreased in the oral cavity of the human subject post-administration of the orally ingestible composition in accordance with the disclosure herein. In some embodiments, the incidence of tooth cavities within the oral cavity of the human subject is reduced following oral administration of the orally ingestible composition.

In some embodiments, the amount of plaque within the oral cavity of the human subject is reduced following oral administration of the orally ingestible composition.

In some embodiments, the gingival score of the oral cavity of the human subject is reduced following oral administration of the orally ingestible composition.

In an embodiment, a method of reducing gastrointestinal discomfort in a human subject is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In further embodiments, the human subject is an adult human male. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to reduce gastrointestinal discomfort in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments, the human subject's total score on the Severity of Dyspepsia Assessment (SODA) scale is reduced following oral administration of the orally ingestible composition.

In some embodiments, the human subject's pain intensity subscore on the SODA scale is reduced following oral administration of the orally ingestible composition.

In some embodiments, the human subject's nonpain symptoms subscore on the SODA scale is reduced following oral administration of the orally ingestible composition.

In an embodiment, a method of improving digestive health in a human subject suffering from acute diarrhea is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In further embodiments, the human subject is an adult human male. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to reduce gastrointestinal discomfort in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments, incidence of diarrhea and/or associated symptoms are reduced in the human subject post-administration. In other embodiments, abdominal pain of the human subject is decreased post-administration. In other embodiments, frequency of defecation of the human subject is decreased post-administration.

In an embodiment, a method of reducing inflammation caused by plaque-induced gingivitis in a human subject is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In further embodiments, the human subject is an adult human male. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to reduce gastrointestinal discomfort in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day. In some embodiments, the activity of the human subject's salivary glutathione peroxidase is decreased post-administration.

In an embodiment, a method of improving protein and amino acid absorption in a human subject is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In further embodiments, the human subject is an adult human male. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to reduce gastrointestinal discomfort in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments, plasma amino acids in the human subject are increased post-administration. In other embodiments, the amino acids comprise branched-chain amino acids. In other embodiments, the subject is further orally administered a protein-containing food. In other embodiments, the protein-containing food comprises whey protein. In some embodiments, the muscle strength of the human subject has a greater increase post-administration of the orally ingestible composition and the protein-containing food in comparison to muscle strength of human subjects consuming only the protein-containing food.

In an embodiment, a method of improving cholesterol levels in a human subject is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In further embodiments, the human subject is an adult human male. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to reduce gastrointestinal discomfort in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments, the human subject's serum HDL is increased post-administration. In other embodiments, the human subject's serum LDL is decreased post-administration. In other embodiments, the human subject's ratio of serum HDL to serum LDL is increased post-administration. In some embodiments, the human subject's total serum cholesterol is decreased post-administration.

In an embodiment, a method of alleviating IBS symptoms in a human subject is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In further embodiments, the human subject is an adult human male. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to reduce gastrointestinal discomfort in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day. In some embodiments, abdominal pain and/or discomfort of the human subject is reduced post-administration. In other embodiments, frequency of complete spontaneous bowel movements is increased post-administration.

In an embodiment, a method of reducing incidence of hepatic encephalopathy in a human subject suffering from liver cirrhosis is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In further embodiments, the human subject is an adult human male. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to reduce gastrointestinal discomfort in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day. In some embodiments, hospital admissions of the human subject are decreased in frequency post-administration. In other embodiments, plasma ammonia levels of the human subject are decreased post-administration.

In an embodiment, a method of improving the immune response of a human subject is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In further embodiments, the human subject is an adult human male. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to reduce gastrointestinal discomfort in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments, at least one of pDC activation, NK cell activation, and anti-viral gene expression is increased in the human subject post-administration. In other embodiments, pathogenesis of an influenza-like illness is reduced or prevented. In other embodiments, expression of at least one of HLA-DR, VIPERIN, IFN-α, and IFN-γ is increased in the human subject post-administration.

In an embodiment, a method of improving or alleviating one or more symptoms of an illness in a human subject is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In further embodiments, the human subject is an adult human male. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to reduce gastrointestinal discomfort in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments, the illness is a common cold, influenza, an influenza-like illness, or a dengue fever-like illness. In other embodiments, the one or more symptoms is at least one of sore throat, cough, feverishness, runny nose, constipation, diarrhea, abdominal pain, sneezing, muscle pain, joint pain, pain behind the eyes, head congestion, loss of appetite, fatigue, and headache.

In an embodiment, a method of improving exercise or work performance in a human subject is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In further embodiments, the human subject is an adult human male. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to reduce gastrointestinal discomfort in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments, the human subject experiences reduced fatigue, lassitude, and/or articular pain post administration. In other embodiments, the human subject experiences increased vigor, general well-being, absolute presenteeism, and/or physical condition post administration.

In an embodiment, a method of maintaining or improving skin condition in a human subject is provided. The method comprises orally administering, to the subject, an orally ingestible composition in accordance with the disclosure herein. In particular embodiments, the subject is a human child under the age of 18. In particular embodiments, the subject is a human adult. In further embodiments, the subject is an adult human female. In further embodiments, the human subject is an adult human male. In some embodiments, the subject is a human teenager between the ages of 13 and 19. In some embodiments, the method comprises orally delivering to the subject an effective amount or concentration of one or more of gold kiwifruit powder, heat-treated Lactococcus lactis, and Bacillus coagulans to reduce gastrointestinal discomfort in the human subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments, a human subject's skin microbiome is maintained post administration. In other embodiments, expression of skin homoeostasis-related genes is increased in the human subject post-administration. In other embodiments, expression of at least one of TGFB1, BD1, BD3, S100A9, CLDN1, CLDN12, OCLN, ZO1, ZO2, and BS1 is increased post administration. In some embodiments, the human subject experiences reduced skin dryness and/or erythema post-administration.

### BRIEF DESCRIPTION OF THE SEVERAL VIEWS OF THE DRAWINGS

The foregoing summary, as well as the following detailed description of embodiments of the compositions and methods disclosed herein, will be better understood when read in conjunction with the appended drawings of exemplary embodiments. It should be understood, however, that the invention is not limited to the precise arrangements and instrumentalities shown.

In the drawings:

FIG. 1 describes a randomized controlled human trial on the effect of kiwifruit capsules consumptions on gut microflora in functionally constipated individuals. FIG. 1A lists a description of tested compositions and dosage amounts. FIG. 1B lists partial results of the study, underscoring the significant increase in abundance of Faecalibacterium prausnitzii in functionally constipated individuals with gold kiwifruit powder.

FIG. 2 summarizes a clinical study on the effect of gold kiwifruit capsules on constipation symptoms and quality of life. FIG. 2A displays improvements of abdominal, rectal, and stool symptoms of constipation. FIG. 2B displays improvement of quality of life symptoms like physical or psychosocial discomfort, worries, concerns, and satisfaction.

FIG. 3 describes the effect of gold kiwifruit capsules on immune response. Values with the same letter are not have statistically significant. FIG. 3A displays increased activation of natural killer cells. FIG. 3B displays increased IFN-γ production in response to tetanus toxoid.

FIG. 4 describes the efficacy of B. coagulans on digestion and constipation in a cohort randomly assigned to placebo vs B. coagulans Unique IS2 capsules (2×109 CFU) daily for 4 weeks. FIG. 3 left panel: mean frequency of spontaneous complete bowl movement; FIG. 3 right panel: mean score of Bristol stool form. (Madempudi et al, 2019).

FIG. 5 describes a clinical study of B. coagulans in the treatment of patients with bacterial vaginosis (BV). FIG. 5A depicts the comparative analysis of BV symptoms in both groups, while FIG. 5B is a summary of the effect of B. coagulans treatment on patients suffering with BV before (day 1) and after (day 90) treatment.

FIG. 6 is an analysis of Gingival Index (GI) scores, Plaque Index (PI) scores, bleeding on probing and Glutathione peroxidase (GPx) activity in placebo (150 mg starch per tables) and treated group (108 CFU B. coagulans), 3 times daily. (Jagadeesh et al, 2017).

FIG. 7 describes the effect of B. coagulans treatment on symptoms of acute diarrhea, including frequency of defecation, consistency of stool, abdominal pain, and total duration of diarrhea per day.

FIG. 8 describes the effect of B. coagulans supplementation on functional constipation in adults compared to lactulose alone and placebo. FIG. 8A shows stool frequency (left panel) and stool consistency (right panel); FIG. 8B shows defecation pain (left panel) and sensation of incomplete evacuation (right panel), and FIG. 8C shows mean abdominal pain.

FIG. 9 describes a double-blind, placebo-controlled study on the effect of B. coagulans supplementation on plasma amino acid levels and muscle strength in resistance trained males consuming whey protein. FIG. 9A shows the effect of probiotic supplementation on muscle strength parameters; FIG. 9B shows the effect of probiotic supplementation on free amino acid levels.

FIG. 10 describes the effect of B. coagulans supplementation on hypercholesterolemia subjects. FIG. 10A shows the effect of B. coagulans supplementation on total serum cholesterol levels; FIG. 10B shows the effect of B. coagulans supplementation on serum triglyceride levels; FIG. 10C shows the effect of B. coagulans supplementation on serum LDL levels; FIG. 10D shows the effect of B. coagulans supplementation on serum HDL levels.

FIG. 11 illustrates scores for mutans streptococci strains (left-hand panels) and lactobacilli strains (right-hand panels) in salivary samples (FIG. 11A) and plaque (FIG. 11B) of children at baseline and after supplementation with B. coagulans (red columns) or placebo (blue columns).

FIG. 12 illustrates the effect of B. coagulans on irritable bowel syndrome (OBS) symptoms in adults (FIG. 12A) and in children (FIG. 12B).

FIG. 13 illustrates the effect of B. coagulans on liver cirrhosis. FIG. 13A displays changes in hospital admissions between treatment and control groups. FIG. 13B displays incidence in cirrhosis complications. FIG. 13C displays changes in plasma ammonia levels. FIG. 13D displays changes in serum bilirubin levels.

FIG. 14 illustrates the effect of B. coagulans on GI symptoms caused by small intestinal bacterial overgrowth.

FIG. 15 illustrates the effect of B. coagulans on inflammation. FIG. 15A displays rat paws before and after injection with carrageenan and after treatment with B. coagulans (L. sporogenes) or B. bifidum. FIG. 15B shows graphically the effect of the treatments on paw thickness from carrageenan-induced edema. FIG. 15C illustrates the effect of the treatments on paw thickness of Wistar rats and percent inhibition of edema achieved by the treatments. FIG. 15D illustrates the improved stair climbing ability of the treated rats. FIG. 15E illustrates the improved motility of treated rats.

FIG. 16 illustrates the effect of L. lactis supplementation on the expression of HLA-DR (FIG. 16A) and transcription levels of anti-viral genes IFNA and VIPERIN in PMBCs (FIG. 16B).

FIG. 17 illustrates the effect of L. lactis supplementation on the increased transcription levels of IFN-α (FIG. 17A) and ISG15 (FIG. 17B) in PMBCs.

FIG. 18 illustrates the effect of L. lactis supplementation on influenza incidence rates in the control group (Town A) and Shizukushi town in which schoolchildren received L. lactis yogurt.

FIG. 19 describes the effect of L. lactis supplementation on cold and influenza symptoms.

FIG. 20 describes the effect of L. lactis supplementation on cumulative incidence days of any severity of cold and flu symptoms (FIG. 20A) and cumulative incidence days of moderate or severe cold and flu symptoms (FIG. 20B).

FIG. 21 illustrates the effect of L. lactis supplementation on cumulative absence days due to URID or GID (FIG. 21A), the cumulative days of symptoms of URID or GID (FIG. 21B) and cumulative days of positive general well-being (FIG. 21C).

FIG. 22 illustrates the effect of L. lactis supplementation on CD86 in the treated group (dashed line) compared to the placebo group (solid line) (FIG. 22A), cumulative days of URTI (FIG. 22B), cumulative days of URTI symptoms (FIG. 22C), and cumulative days of systemic symptoms (FIG. 22D).

FIG. 23 describes the effect of L. lactis supplementation on work presenteeism (FIG. 23A), mood states (FIG. 23B), and cumulative days of cold symptoms and physical condition (FIG. 23C).

FIG. 24 describes the effect of L. lactis supplementation on cumulative incidence days of dengue fever-like symptoms.

FIG. 25 illustrates the effect of L. lactis supplementation on the skin microbiome for Staphylococcus epidermis (FIG. 25A), Staphylococcus pasteuri (FIG. 25B), Staphylococcus roseiflava (FIG. 25C), and Propionibacterium acnes (FIG. 25D). FIG. 25E describes the effect of L. lactis supplementation on relative expression levels of skin homeostasis related genes. FIG. 25F describes the effect of L. lactis supplementation on skin condition.

FIG. 26 illustrates the effect of L. lactis supplementation on the genus (FIG. 26A) and species (FIG. 26B) of bacteria in the skin microbiota. FIG. 26C describes the effect of L. lactis supplementation on relative expression of skin-homeostasis-related genes.

### DETAILED DESCRIPTION OF THE INVENTION

Oral supplements and foods including probiotics, beneficial live microorganisms that confer health benefits on their “host” organisms (e.g., humans), have gained popularity as nutritional and health aids. However, to confer a substantial and/or lasting health benefit on those using them, the probiotics must be administered in an adequate amount and have an environment in which they can persist within the subject. It is often difficult to provide an adequate “stabilized” microbiome environment within a “host” organism to reap the full benefits of probiotic supplementation or consumption.

The interactions between probiotics, prebiotics, and postbiotics have recently garnered attention for the potential to address the issues associated with reaping the benefits of probiotics. Prebiotics may be considered to be probiotic “food”; that is, they are substrates selectively used by probiotic microorganisms in their life cycle maintenance, helping the probiotics survive during digestion to be delivered to the appropriate area of the digestive system. Prebiotics may also be used by the probiotic microorganisms to confer health benefits to the “host” organism. Most, if not, all prebiotic supplements utilize prebiotic fibers like fructooligosaccharides (FOS), xylooligosaccharides (XOS), galacto-oligosaccharides (GOS), or inulin due to low dosage requirements. However, these prebiotics are not considered FODMAP-friendly, as they are fermentable oligosaccharides which can cause unwanted GI side effects (e.g., bloating, gas).

By contrast, postbiotics may comprise inanimate or dead microorganisms, their respective parts, or waste byproducts produced by probiotic microorganisms that create a healthy microenvironment for probiotic or beneficial microorganisms to flourish. Postbiotics in the form of dead microorganisms may also confer similar health benefits to that of probiotics, but are not at risk of biological instability, since they are already non-viable. It is believed that the interplay between prebiotics, probiotics, and postbiotics may synergistically enhance the beneficial function each is known to confer separately, as the addition of pre- and postbiotics to probiotics is believed to stabilize the life cycle processes of the probiotic microorganisms.

Here, the inventors have combined pre-, pro-, and postbiotics in surprisingly efficacious manners, yielding beneficial nutritive compositions that demonstrate an enhanced, synergistic effect when consumed by a subject, leading to increased health benefits for the subject. The inventors have also developed surprisingly effective methods of using these beneficial compositions for increasing the health of subjects taking these compositions orally. Moreover, the inventors have used FODMAP-friendly prebiotics in compositions according to the disclosure herein to avoid the issues associated with typical prebiotic-containing foods and supplements.

### I. COMPOSITIONS

In an aspect of the invention, an orally ingestible nutritional composition for improving health is provided. The composition comprises an amount of gold kiwifruit powder (e.g., a prebiotic), an amount of Bacillus coagulans (e.g., a probiotic), and an amount of heat-treated Lactococcus lactis (e.g., a postbiotic). The gold kiwifruit powder within the composition may include or consist essentially of Livaux®. The Bacillus coagulans within the composition may include or consist essentially of UNIQUE IS-2®. The heat-treated Lactococcus lactis within the composition may include or consist essentially of IMMUSE™.

In further embodiments, an individual oral dosage form may comprise between about 75 mg and 700 mg gold kiwifruit powder; between about 5 mg and 80 mg heat-treated Lactococcus lactis; and between about 5 mg and 200 mg Bacillus coagulans. In further embodiments, an individual oral dosage form may comprise between about 75 mg and 100 mg gold kiwifruit powder; between about 5 mg and 15 mg heat-treated Lactococcus lactis; and between about 10 mg and 20 mg Bacillus coagulans. In further embodiments, an individual oral dosage form may comprise between about 100 mg and 200 mg gold kiwifruit powder; between about 15 mg and 25 mg heat-treated Lactococcus lactis; and between about 20 mg and 35 mg Bacillus coagulans. In further embodiments, an individual oral dosage form may comprise between about 200 mg and 300 mg gold kiwifruit powder; between about 25 mg and 45 mg heat-treated Lactococcus lactis; and between about 35 mg and 65 mg Bacillus coagulans. In further embodiments, an individual oral dosage form may comprise between about 300 mg and 500 mg gold kiwifruit powder; between about 45 mg and 70 mg heat-treated Lactococcus lactis; and between about 65 mg and 100 mg Bacillus coagulans. In further embodiments, an individual oral dosage form may comprise between about 500 mg and 700 mg gold kiwifruit powder; between about 70 mg and 100 mg heat-treated Lactococcus lactis; and between about 100 mg and 150 mg Bacillus coagulans.

In particular embodiments, the individual oral dosage form comprises about 75 mg gold kiwifruit powder; about 10.8 mg heat-treated Lactococcus lactis; and about 15 mg Bacillus coagulans. In particular embodiments, the individual oral dosage form comprises about 125 mg gold kiwifruit powder; about 18 mg heat-treated Lactococcus lactis; and about 25 mg Bacillus coagulans. In particular embodiments, the individual oral dosage form comprises about 250 mg gold kiwifruit powder; about 36 mg heat-treated Lactococcus lactis; and about 50 mg Bacillus coagulans. In particular embodiments, the individual oral dosage form comprises about 500 mg gold kiwifruit powder; about 72 mg heat-treated Lactococcus lactis; and about 100 mg Bacillus coagulans. In particular embodiments, the individual oral dosage form comprises about 700 mg gold kiwifruit powder; about 100 mg heat-treated Lactococcus lactis; and about 140 mg Bacillus coagulans.

According to certain embodiments of the inventive composition, the compositions are formulated such that they cause improvement in health markers within a subject consuming them (e.g., a demonstrable improvement in digestive health). Health improvements may be measured by improvement in symptoms of particular disorders (e.g., reduction in the amount of Streptococcus mutans within the oral cavity), or increase in markers of health (e.g., increase in the amount of Faecalibacterium prausnitzii within a digestive tract).

In a further embodiment, the composition includes concentrations or amounts of the pre-, pro-, and postbiotics that are therapeutically effective to cause improvement in health markers within the subject.

In a preferred embodiment of the composition, the composition includes gold kiwifruit powder as a prebiotic. Gold kiwifruit powder possesses well-demonstrated tolerability in vivo and is FODMAP-friendly. It is also shown to increase the presence of Faecalibacterium prausnitzii in the microbiome, a beneficial gut probiotic microorganism. Gold kiwifruit powder has also been shown to improve gut transit, contribute to fecal bulking, and increase mucus production in the gut. Clinically or therapeutically effective amounts, or amounts sufficient to cause improvement in health markers within the subject, of the gold kiwifruit powder in compositions according to the invention herein include from about 75 mg to about 2800 mg of gold kiwifruit powder per daily total dose, from about 75 mg to about 2500 mg per daily total dose; from about 75 mg to about 2250 mg per daily total dose; from about 75 mg to about 2000 mg per daily total dose; from about 75 mg to about 1750 mg per daily total dose; from about 75 mg to about 1500 mg per daily total dose; from about 75 mg to about 1250 mg per daily total dose; from about 75 mg to about 1000 mg per daily total dose; from about 75 mg to about 750 mg per daily total dose; from about 75 mg to about 500 mg per daily dose; or from about 75 mg to about 250 mg per daily total dose. The gold kiwifruit powder may comprise Livaux®, which is a powdered formulation from cold-processed Actinidia chinensis var. chinensis ‘Zesy002’ gold-fleshed kiwifruit.

In particular embodiments, clinically or therapeutically effective amounts, or amounts sufficient to cause improvement in health markers within the subject, of the gold kiwifruit powder in compositions according to the invention herein include about 2800 mg of gold kiwifruit powder per daily total dose, about 2500 mg per daily total dose; about 2250 mg per daily total dose; about 2000 mg per daily total dose; about 1750 mg per daily total dose; about 1500 mg per daily total dose; about 1250 mg per daily total dose; about 1000 mg per daily total dose; about 750 mg per daily total dose; about 500 mg per daily dose; about 450 mg per daily total dose; about 400 mg per daily total dose; about 350 mg per daily total dose; about 300 mg per daily total dose; about 250 mg per daily total dose; about 225 mg per daily total dose; about 200 mg per daily total dose; about 175 mg per daily total dose; about 150 mg per daily total dose; about 125 mg per daily total dose; about 100 mg per daily total dose; or about 75 mg per daily total dose.

In a preferred embodiment of the composition, the composition includes Bacillus coagulans as a probiotic. In particularly preferred embodiments, the B. coagulans comprises UNIQUE IS-2®. UNIQUE IS-2® is a specific strain of B. coagulans that has been shown to improve gut, oral, and immune health across multiple therapeutic endpoints and in different human populations, including in children and teenagers.

Clinically or therapeutically effective amounts, or amounts sufficient to cause improvement in health markers within the subject, of the Bacillus coagulans in compositions according to the invention herein include from about 5 mg to about 800 mg of Bacillus coagulans per daily total dose, from about 5 mg to about 700 mg per daily total dose; from about 5 mg to about 600 mg per daily total dose; from about 5 mg to about 500 mg per daily total dose; from about 5 mg to about 400 mg per daily total dose; from about 5 mg to about 300 mg per daily total dose; from about 5 mg to about 250 mg per daily total dose; from about 5 mg to about 200 mg per daily total dose; from about 5 mg to about 150 mg per daily total dose; from about 5 mg to about 140 mg per daily total dose; from about 5 mg to about 130 mg per daily total dose; from about 5 mg to about 120 mg per daily total dose; from about 5 mg to about 110 mg per daily total dose; from about 5 mg to about 100 mg per daily total dose; from about 5 mg to about 90 mg per daily total dose; from about 5 mg to about 80 mg per daily total dose; from about 5 mg to about 70 mg per daily total dose; from about 5 mg to about 60 mg per daily total dose; from about 5 mg to about 50 mg per daily total dose; from about 5 mg to about 40 mg per daily total dose; from about 5 mg to about 30 mg per daily total dose; or from about 5 mg to about 20 mg per daily total dose. Clinically or therapeutically effective amounts, or amounts sufficient to cause improvement in health markers within the subject, of the Bacillus coagulans in compositions according to the invention herein include between about 500 million CFUs and 80 billion CFUs Bacillus coagulans per daily total dose, from about 500 million CFUs to about 70 billion CFUs Bacillus coagulans per daily total dose, from about 500 million CFUs to about 60 billion CFUs Bacillus coagulans per daily total dose, from about 500 million CFUs to about 50 billion CFUs Bacillus coagulans per daily total dose, from about 500 million CFUs to about 40 billion CFUs Bacillus coagulans per daily total dose, from about 500 million CFUs to about 30 billion CFUs Bacillus coagulans per daily total dose, from about 500 million CFUs to about 20 billion CFUs Bacillus coagulans per daily total dose, from about 500 million CFUs to about 19 billion CFUs Bacillus coagulans per daily total dose, from about 500 million CFUs to about 18 billion CFUs Bacillus coagulans per daily total dose, from about 500 million CFUs to about 17 billion CFUs Bacillus coagulans per daily total dose, from about 500 million CFUs to about 16 billion CFUs Bacillus coagulans per daily total dose, from about 500 million CFUs to about 15 billion CFUs Bacillus coagulans per daily total dose, from about 500 million CFUs to about 14 billion CFUs Bacillus coagulans per daily total dose, from about 500 million CFUs to about 13 billion CFUs Bacillus coagulans per daily total dose, from about 500 million CFUs to about 12 billion CFUs Bacillus coagulans per daily total dose, from about 500 million CFUs to about 11 billion CFUs Bacillus coagulans per daily total dose, from about 500 million CFUs to about 10 billion CFUs Bacillus coagulans per daily total dose, from about 500 million CFUs to about 9 billion CFUs Bacillus coagulans per daily total dose, from about 500 million CFUs to about 8 billion CFUs Bacillus coagulans per daily total dose, from about 500 million CFUs to about 7 billion CFUs Bacillus coagulans per daily total dose, from about 500 million CFUs to about 6 billion CFUs Bacillus coagulans per daily total dose, from about 500 million CFUs to about 5 billion CFUs Bacillus coagulans per daily total dose, from about 500 million CFUs to about 4 billion CFUs Bacillus coagulans per daily total dose, from about 500 million CFUs to about 3 billion CFUs Bacillus coagulans per daily total dose, from about 500 million CFUs to about 2 billion CFUs Bacillus coagulans per daily total dose, from about 750 million CFUs to about 10 billion CFUs Bacillus coagulans per daily total dose, from about 2 billion CFUs to about 8 billion CFUs Bacillus coagulans per daily total dose, from about 3 billion CFUs to about 6 billion CFUs Bacillus coagulans per daily total dose, from about 4.5 billion CFUs to about 5.5 billion CFUs Bacillus coagulans per daily total dose, from about 1 billion CFUs to about 15 billion CFUs Bacillus coagulans per daily total dose, from about 1 billion CFUs to about 10 billion CFUs Bacillus coagulans per daily total dose, from about 2 billion CFUs to about 8 billion CFUs Bacillus coagulans per daily total dose, from about 4 billion CFUs to about 6 billion CFUs Bacillus coagulans per daily total dose, about 500 million CFUs, about 750 million CFUS, about 1 billion CFUs, about 1.25 billion CFUs, about 1.5 billion CFUS, about 1.75 billion CFUs, about 2 billion CFUs, about 2.25 billion CFUs, about 2.5 billion CFUS, about 2.75 billion CFUs, about 3 billion CFUs, about 3.25 billion CFUs, about 3.5 billion CFUS, about 3.75 billion CFUs, about 4 billion CFUs, about 4.25 billion CFUs, about 4.5 billion CFUS, about 4.75 billion CFUs, about 5 billion CFUs, about 5.25 billion CFUs, about 5.5 billion CFUS, about 5.75 billion CFUs, about 6 billion CFUs, about 7 billion CFUs, about 8 billion CFUs, about 9 billion CFUs, about 10 billion CFUs, about 11 billion CFUs, about 12 billion CFUs, about 13 billion CFUs, about 14 billion CFUs, about 15 billion CFUs, about 16 billion CFUs per daily total dose, about 17 billion CFUs per daily total dose, about 18 billion CFUs per daily total dose, about 19 billion CFUs per daily total dose, about 20 billion CFUs per daily total dose, about 30 billion CFUs per daily total dose, about 40 billion CFUs per daily total dose, about 50 billion CFUs per daily total dose, about 60 billion CFUs per daily total dose, about 70 billion CFUs per daily total dose, or about 80 billion CFUs per daily total dose. The B. coagulans may comprise UNIQUE IS-2®.

In particular embodiments, clinically or therapeutically effective amounts, or amounts sufficient to cause improvement in health markers within the subject, of the Bacillus coagulans in compositions according to the invention herein include about 800 mg of Bacillus coagulans per daily total dose, about 700 mg of Bacillus coagulans per daily total dose, about 600 mg of Bacillus coagulans per daily total dose, about 500 mg of Bacillus coagulans per daily total dose, about 400 mg of Bacillus coagulans per daily total dose, about 300 mg of Bacillus coagulans per daily total dose, about 200 mg of Bacillus coagulans per daily total dose, about 190 mg of Bacillus coagulans per daily total dose, about 180 mg of Bacillus coagulans per daily total dose, about 170 mg of Bacillus coagulans per daily total dose, about 160 mg of Bacillus coagulans per daily total dose, about 150 mg per daily total dose; about 140 mg per daily total dose; about 130 mg per daily total dose; about 120 mg per daily total dose; about 110 mg per daily total dose; about 100 mg per daily total dose; about 90 mg per daily total dose; about 80 mg per daily total dose; about 70 mg per daily total dose; about 60 mg per daily total dose; about 50 mg per daily total dose; about 40 mg per daily total dose; about 30 mg per daily total dose; about 20 mg per daily total dose; about 15 mg per daily total dose; about 10 mg per daily total dose; or about 5 mg per daily total dose. The B. coagulans may comprise UNIQUE IS-2®.

In a preferred embodiment of the composition, the composition includes heat-treated (i.e., killed) Lactococcus lactis as a postbiotic. In particularly preferred embodiments, the heat-treated Lactococcus lactis comprises IMMUSE™. IMMUSE™ has been shown to support immunomodulatory effects across multiple therapeutic endpoints and in different human populations and is a particular heat-treated Lactococcus lactis strain known as Lactococcus lactis strain Plasma (“LC-Plasma”).

Clinically or therapeutically effective amounts, or amounts sufficient to cause beneficial immunomodulatory effects within the subject, of the Lactococcus lactis in compositions according to the invention herein include from about 5 mg to about 320 mg of Lactococcus lactis per daily total dose; from about 5 mg to about 300 mg per daily total dose; from about 5 mg to about 250 mg per daily total dose; from about 5 mg to about 200 mg per daily total dose; from about 5 mg to about 175 mg per daily total dose; from about 5 mg to about 165 mg per daily total dose; from about 5 mg to about 155 mg per daily total dose; from about 5 mg to about 145 mg per daily total dose; from about 5 mg to about 135 mg per daily total dose; from about 5 mg to about 125 mg per daily total dose; from about 5 mg to about 115 mg per daily total dose; from about 5 mg to about 105 mg per daily total dose; from about 5 mg to about 95 mg per daily total dose; from about 5 mg to about 85 mg per daily total dose; from about 5 mg to about 75 mg per daily total dose; from about 5 mg to about 65 mg per daily total dose; from about 5 mg to about 55 mg per daily total dose; from about 15 mg to about 45 mg per daily total dose; from about 25 mg to about 40 mg per daily total dose; or from about 30 mg to about 40 mg per daily total dose.

In particular embodiments, clinically or therapeutically effective amounts, or amounts sufficient to cause beneficial immunomodulatory effects within the subject, of the Lactococcus lactis in compositions according to the invention herein include about 320 mg of Lactococcus lactis per daily total dose, about 300 mg of Lactococcus lactis per daily total dose, about 275 mg of Lactococcus lactis per daily total dose, about 250 mg of Lactococcus lactis per daily total dose, about 225 mg of Lactococcus lactis per daily total dose, about 200 mg of Lactococcus lactis per daily total dose, about 175 mg of Lactococcus lactis per daily total dose, about 150 mg of Lactococcus lactis per daily total dose, about 135 mg per daily total dose; about 125 mg per daily total dose; about 115 mg per daily total dose; about 105 mg per daily total dose; about 100 mg per daily total dose; about 95 mg per daily total dose; about 90 mg per daily total dose; about 85 mg per daily total dose; about 80 mg per daily total dose; about 75 mg per daily total dose; about 70 mg per daily total dose; about 65 mg per daily total dose; about 60 mg per daily total dose; about 55 mg per daily total dose; about 50 mg per daily total dose; about 45 mg per daily total dose; about 40 mg per daily total dose; about 35 mg per daily total dose; about 30 mg per daily total dose; about 25 mg per daily total dose; about 20 mg per daily total dose; about 15 mg per daily total dose; about 10 mg per daily total dose; or about 5 mg per daily total dose. The Lactococcus lactis may comprise IMMUSE™.

Compositions according to embodiments of the invention may include combinations of components each in clinically or therapeutically effective amounts. For example, in some compositional embodiments, each of the gold kiwifruit powder, the B. coagulans, and the Lactococcus lactis may be included in therapeutically effective amounts in composition.

In some embodiments, components of the inventive composition may be combined at a w/w ratio of between about 2:1 and about 15:1 gold kiwifruit powder: Bacillus coagulans. In some embodiments, components of the inventive composition may be combined at a w/w ratio of between about 2:1 and about 10:1 gold kiwifruit powder: Bacillus coagulans. In some embodiments, components of the inventive composition may be combined at a w/w ratio of between about 3:1 and about 9:1 gold kiwifruit powder: Bacillus coagulans. In some embodiments, components of the inventive composition may be combined at a w/w ratio of between about 4:1 and about 8:1 gold kiwifruit powder: Bacillus coagulans. In some embodiments, components of the inventive composition may be combined at a w/w ratio of between about 4:1 and about 7:1 gold kiwifruit powder: Bacillus coagulans. In some embodiments, components of the inventive composition may be combined at a w/w ratio of between about 4:1 and about 6:1 gold kiwifruit powder: Bacillus coagulans. In some embodiments, components of the inventive composition may be combined at a w/w ratio of between about 4.5:1 and about 5.5:1 gold kiwifruit powder:Bacillus coagulans.

In some embodiments, components of the inventive composition may be combined at a w/w ratio of between about 5:1 and about 20:1 gold kiwifruit powder:Lactococcus lactis. In some embodiments, components of the inventive composition may be combined at a w/w ratio of between about 5:1 and about 15:1 gold kiwifruit powder:Lactococcus lactis. In some embodiments, components of the inventive composition may be combined at a w/w ratio of between about 5:1 and about 10:1 gold kiwifruit powder:Lactococcus lactis. In some embodiments, components of the inventive composition may be combined at a w/w ratio of between about 5:1 and about 9:1 gold kiwifruit powder:Lactococcus lactis. In some embodiments, components of the inventive composition may be combined at a w/w ratio of between about 3:1 and about 8:1 gold kiwifruit powder:Lactococcus lactis. In some embodiments, components of the inventive composition may be combined at a w/w ratio of between about 4:1 and about 8:1 gold kiwifruit powder:Lactococcus lactis. In some embodiments, components of the inventive composition may be combined at a w/w ratio of between about 5:1 and about 8:1 gold kiwifruit powder:Lactococcus lactis.

In some embodiments, components of the inventive composition may be combined at a w/w ratio of between about 1:1 and about 5:1: Bacillus coagulans:Lactococcus lactis. In some embodiments, components of the inventive composition may be combined at a w/w ratio of between about 1:1 and about 4.5:1: Bacillus coagulans:Lactococcus lactis. In some embodiments, components of the inventive composition may be combined at a w/w ratio of between about 1:1 and about 4:1: Bacillus coagulans:Lactococcus lactis. In some embodiments, components of the inventive composition may be combined at a w/w ratio of between about 1:1 and about 3.5:1: Bacillus coagulans:Lactococcus lactis. In some embodiments, components of the inventive composition may be combined at a w/w ratio of between about 1:1 and about 3:1: Bacillus coagulans:Lactococcus lactis. In some embodiments, components of the inventive composition may be combined at a w/w ratio of between about 1:1 and about 3.5:1: Bacillus coagulans:Lactococcus lactis. In some embodiments, components of the inventive composition may be combined at a w/w ratio of between about 1:1 and about 2.5:1: Bacillus coagulans:Lactococcus lactis. In some embodiments, components of the inventive composition may be combined at a w/w ratio of between about 1:1 and about 2:1: Bacillus coagulans:Lactococcus lactis.

In some embodiments, components of the inventive composition may be combined at a w/w ratio of between about 5:1 and about 20:1 gold kiwifruit powder: Bacillus coagulans. In some embodiments, components of the inventive composition may be combined at a w/w ratio of between about 7:1 and about 18:1 gold kiwifruit powder: Bacillus coagulans. In some embodiments, components of the inventive composition may be combined at a w/w ratio of between about 10:1 and about 15:1 gold kiwifruit powder:Bacillus coagulans. In some embodiments, components of the inventive composition may be combined at a w/w ratio of between about 12:1 and about 14:1 gold kiwifruit powder:Bacillus coagulans. In some embodiments, components of the inventive composition may be combined at a w/w ratio of between about 15:1 and about 20:1 gold kiwifruit powder:Bacillus coagulans. In some embodiments, components of the inventive composition may be combined at a w/w ratio of between about 12.5:1 and about 17.5:1 gold kiwifruit powder:Bacillus coagulans. In some embodiments, components of the inventive composition may be combined at a w/w ratio of between about 7.5:1 and about 12.5:1 gold kiwifruit powder:Bacillus coagulans.

In some embodiments, components of the inventive composition may be combined at a w/w ratio of between about 3:1 and about 7:1 gold kiwifruit powder:Lactococcus lactis. In some embodiments, components of the inventive composition may be combined at a w/w ratio of between about 3.5:1 and about 6.5:1 gold kiwifruit powder:Lactococcus lactis. In some embodiments, components of the inventive composition may be combined at a w/w ratio of between about 4:1 and about 6:1 gold kiwifruit powder: Lactococcus lactis. In some embodiments, components of the inventive composition may be combined at a w/w ratio of between about 4.5:1 and about 5.5:1 gold kiwifruit powder:Lactococcus lactis. In some embodiments, components of the inventive composition may be combined at a w/w ratio of between about 5:1 and about 7:1 gold kiwifruit powder:Lactococcus lactis. In some embodiments, components of the inventive composition may be combined at a w/w ratio of between about 4.5:1 and about 6.5:1 gold kiwifruit powder:Lactococcus lactis. In some embodiments, components of the inventive composition may be combined at a w/w ratio of between about 4:1 and about 7:1 gold kiwifruit powder:Lactococcus lactis.

In some embodiments, components of the inventive composition may be combined at a w/w ratio of between about 1.5:1 and about 3.5:1: Bacillus coagulans:Lactococcus lactis. In some embodiments, components of the inventive composition may be combined at a w/w ratio of between about 1.75:1 and about 3.25:1: Bacillus coagulans:Lactococcus lactis. In some embodiments, components of the inventive composition may be combined at a w/w ratio of between about 2:1 and about 3:1: Bacillus coagulans:Lactococcus lactis. In some embodiments, components of the inventive composition may be combined at a w/w ratio of between about 2.25:1 and about 2.75:1: Bacillus coagulans:Lactococcus lactis. In some embodiments, components of the inventive composition may be combined at a w/w ratio of between about 2.5:1 and about 3.5:1: Bacillus coagulans:Lactococcus lactis. In some embodiments, components of the inventive composition may be combined at a w/w ratio of between about 1.5:1 and about 3:1: Bacillus coagulans:Lactococcus lactis. In some embodiments, components of the inventive composition may be combined at a w/w ratio of between about 2:1 and about 2.5:1: Bacillus coagulans:Lactococcus lactis. In some embodiments, components of the inventive composition may be combined at a w/w ratio of between about 2:1 and about 3.5:1: Bacillus coagulans:Lactococcus lactis.

Compositions according to certain embodiments herein may be formulated as solid or liquid dosage forms. Dosage forms may be formulated for oral delivery. Oral delivery methods for compositions according to certain embodiments of the invention herein may include gummies, tablets, capsules, liquids, suspensions, chewables, soft gels, sachets, powders, syrups, liquid suspensions, emulsions, or other solutions. In particular embodiments, the orally ingestible composition is in the form of an individual dosage form. The individual dosage form may take the form of a gummy, a tablet, a capsule, or a chew. In particular embodiments, the individual dosage form is a gummy.

### II. METHODS

In an embodiment, a method of increasing the amount of Faecalibacterium prausnitzii within a digestive tract of a subject is provided. The method comprises orally administering, to the subject, an orally ingestible composition in accordance with the disclosure herein. In particular embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In further embodiments, the subject is an adult human female. In particular embodiments, the subject is a human teenager between the ages of 13 and 19. In some embodiments of increasing the amount of Faecalibacterium prausnitzii within a digestive tract of a subject, the method comprises orally delivering to the subject an effective amount or concentration of one or more of gold kiwifruit powder, Lactococcus lactis, and Bacillus coagulans to achieve an increase in the amount of Faecalibacterium prausnitzii within the digestive tract of the subject. In some embodiments of the method of increasing the amount of Faecalibacterium prausnitzii within a digestive tract of a subject, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In an embodiment, a method of reducing constipation or relieving symptoms of constipation in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In particular embodiments, the subject is a human teenager between the ages of 13 and 19.

In some embodiments, the method of reducing constipation or relieving symptoms of constipation in a subject comprises orally delivering to the subject an effective amount or concentration of one or more of gold kiwifruit powder, Lactococcus lactis, and Bacillus coagulans to achieve a reduction in constipation or relief of symptoms of constipation in the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of reducing constipation or relieving symptoms of constipation in a subject, the subject's frequency of defecation is increased post-administration of the orally ingestible composition. In some embodiments, the subject's frequency of defecation is increased post-administration to at least three bowel movements per week.

In some embodiments of the method of reducing constipation or relieving symptoms of constipation in a subject, the subject's frequency of complete spontaneous bowel movements is increased. In some embodiments, the subject's frequency of complete spontaneous bowel movements is increased post-administration to at least three complete spontaneous bowel movements per week.

In some embodiments of the method of reducing constipation or relieving symptoms of constipation in a subject, the subject's stools are softened and/or improved in consistency post-administration of the orally ingestible composition as measured by the Bristol stool form scale. In some embodiments, the subject's stool consistency post-administration achieves a 3 or 4 on the Bristol stool form scale.

In an embodiment, a method of improving immune health in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In particular embodiments, the subject is a human teenager between the ages of 13 and 19.

In some embodiments, the method of improving immune health in a subject comprises orally delivering to the subject an effective amount or concentration of one or more of gold kiwifruit powder, Lactococcus lactis, and Bacillus coagulans to achieve an improvement in immune health of a subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of improving immune health in a subject, the incidence frequency of colds and/or cold symptoms in the subject are decreased post-administration of the orally ingestible composition. In some embodiments, the subject's frequency of experiencing a cold and/or exhibiting cold symptoms is reduced by at least 5%, at least 10%, at least 15%, at least 20%, at least 25%, at least 30%, at least 35%, at least 40%, at least 45%, or at least 50% post-administration of the orally ingestible composition.

In some embodiments of the method of improving immune health in a subject, the incidence frequency of upper respiratory infections and/or symptoms thereof in the subject are decreased post-administration of the orally ingestible composition. In some embodiments, the subject's frequency of experiencing upper respiratory infections and/or symptoms thereof is reduced by at least 5%, at least 10%, at least 15%, at least 20%, at least 25%, at least 30%, at least 35%, at least 40%, at least 45%, or at least 50% post-administration of the orally ingestible composition.

In some embodiments of the method of improving immune health in a subject, the incidence frequency of gastrointestinal (GI) infections and/or symptoms thereof in the subject are decreased post-administration of the orally ingestible composition. In some embodiments, the subject's frequency of experiencing GI infections and/or symptoms thereof is reduced by at least 5%, at least 10%, at least 15%, at least 20%, at least 25%, at least 30%, at least 35%, at least 40%, at least 45%, or at least 50% post-administration of the orally ingestible composition.

In an embodiment, a method of improving oral health in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In further embodiments, the subject is an adult human female.

In some embodiments, the method of improving oral health in a subject comprises orally delivering to the subject an effective amount or concentration of one or more of gold kiwifruit powder, Lactococcus lactis, and Bacillus coagulans to achieve an improvement in oral health of a subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of improving oral health in a subject, the amount of Streptococcus mutans within the oral cavity of the subject is reduced post-administration of the orally ingestible composition. In some embodiments, the amount of Streptococcus mutans within the oral cavity of the subject is reduced by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving oral health in a subject, the incidence frequency of tooth cavities within the oral cavity of the subject is decreased post-administration of the orally ingestible composition. In some embodiments, the subject's frequency of experiencing tooth cavities is reduced by at least 5%, at least 10%, at least 15%, at least 20%, at least 25%, at least 30%, at least 35%, at least 40%, at least 45%, or at least 50% post-administration of the orally ingestible composition.

In some embodiments of the method of improving oral health in a subject, the amount of plaque within the oral cavity of the subject is reduced post-administration of the orally ingestible composition. In some embodiments, the amount of plaque within the oral cavity of the subject is reduced by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving oral health in a subject, the gingival score of the oral cavity of the subject is decreased post-administration of the orally ingestible composition. In some embodiments, the subject's gingival score is reduced by at least 5%, at least 10%, at least 15%, at least 20%, at least 25%, at least 30%, at least 35%, at least 40%, at least 45%, or at least 50% post-administration of the orally ingestible composition.

In an embodiment, a method of reducing bacterial vaginosis symptoms in a human female subject is provided. The method comprises orally administering, to the human female subject, an orally ingestible composition in accordance with the disclosure herein. In particular embodiments, the subject is a human female child under the age of 18. In particular embodiments, the subject is a human female child between the ages of 0 and 3. In particular embodiments, the subject is a human female child between the ages of 0 and 5. In particular embodiments, the subject is a human female child between the ages of 1 and 5. In particular embodiments, the subject is a human female child between the ages of 6 and 10. In particular embodiments, the subject is a human female child between the ages of 11 and 15. In particular embodiments, the subject is a human female child between the ages of 15 and 17. In particular embodiments, the subject is an adult human female. In some embodiments, the subject is a human female teenager between the ages of 13 and 19

In some embodiments, the method of reducing bacterial vaginosis symptoms comprises orally delivering to the subject an effective amount or concentration of one or more of gold kiwifruit powder, Lactococcus lactis, and Bacillus coagulans to reduce bacterial vaginosis symptoms in the subject. In some embodiments, the method of reducing bacterial vaginosis symptoms in a human female subject comprises orally delivering to the subject an effective amount or concentration of one or more of gold kiwifruit powder, Lactococcus lactis, and Bacillus coagulans to achieve a reduction in the bacterial vaginosis symptoms of the human female subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day. In some embodiments, the human female subject is undergoing antibiotic treatment for bacterial vaginosis.

In an embodiment, a method of reducing gastrointestinal discomfort in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In some embodiments, the subject is a human teenager between the ages of 13 and 19.

In some embodiments, the method of reducing gastrointestinal discomfort in a subject comprises orally delivering to the subject an effective amount or concentration of one or more of gold kiwifruit powder, Lactococcus lactis, and Bacillus coagulans to achieve a reduction in gastrointestinal discomfort of the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of reducing gastrointestinal discomfort in a subject, the subject's total score on the Severity of Dyspepsia Assessment (SODA) scale is reduced post-administration of the orally ingestible composition. In some embodiments, the subject's total score on the SODA scale is reduced by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of reducing gastrointestinal discomfort in a subject, the subject's nonpain symptoms subscore on the SODA scale is reduced post-administration of the orally ingestible composition. In some embodiments, the subject's nonpain symptoms subscore on the SODA scale is reduced by at least 5%, at least 10%, at least 15%, at least 20%, at least 25%, at least 30%, at least 35%, at least 40%, at least 45%, or at least 50% post-administration of the orally ingestible composition.

In an embodiment, a method of improving digestive health in a subject suffering from acute diarrhea is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In some embodiments, the subject is a human teenager between the ages of 13 and 19.

In some embodiments, the method of improving digestive health in a subject suffering from acute diarrhea comprises orally delivering to the subject an effective amount or concentration of one or more of gold kiwifruit powder, Lactococcus lactis, and Bacillus coagulans to achieve an improvement in digestive health of the subject suffering from acute diarrhea. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of improving digestive health in a subject, the subject's incidence of diarrhea and/or associated symptoms are reduced post-administration of the orally ingestible composition. In some embodiments, the subject's incidence of diarrhea and/or associated symptoms are reduced by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving digestive health in a subject, the subject's abdominal pain is decreased post-administration of the orally ingestible composition. In some embodiments, the subject's abdominal pain is decreased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving digestive health in a subject, the subject's frequency of defecation is decreased post-administration of the orally ingestible composition. In some embodiments, the subject's frequency of defecation is decreased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In an embodiment, a method of reducing inflammation caused by plaque-induced gingivitis in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In some embodiments, the subject is a human teenager between the ages of 13 and 19.

In some embodiments, the method of reducing inflammation caused by plaque-induced gingivitis in a subject comprises orally delivering to the subject an effective amount or concentration of one or more of gold kiwifruit powder, Lactococcus lactis, and Bacillus coagulans to achieve a reduction of inflammation caused by plaque-induced gingivitis of the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of reducing inflammation caused by plaque-induced gingivitis in a subject, activity of the subject's salivary glutathione peroxidase is decreased post-administration of the orally ingestible composition. In some embodiments, the activity of the subject's salivary glutathione peroxidase is decreased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In an embodiment, a method of improving protein and amino acid absorption in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In some embodiments, the subject is a human teenager between the ages of 13 and 19.

In some embodiments, the method of improving protein and amino acid absorption in a subject comprises orally delivering to the subject an effective amount or concentration of one or more of gold kiwifruit powder, Lactococcus lactis, and Bacillus coagulans to achieve an improvement in protein and amino acid absorption of the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of improving protein and amino acid absorption in a subject, the subject's plasma amino acids are increased post-administration of the orally ingestible composition. In some embodiments, the subject's plasma amino acids are increased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving protein and amino acid absorption in a subject, the subject's muscle strength has a greater increase post-administration of the orally ingestible composition and a protein-containing food in comparison to muscle strength of human subjects consuming only the protein-containing food. In some embodiments, the subject's muscle strength is increased greater post-administration of the orally ingestible composition by at least 1%, at least 3%, at least 5%, at least 10%, at least 15%, at least 20%, at least 25%, at least 30%, at least 35%, at least 40%, at least 45%, or at least 50% in comparison to increased muscle strength of human subjects consuming only the protein-containing food.

In an embodiment, a method of improving cholesterol levels in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In some embodiments, the subject is a human teenager between the ages of 13 and 19.

In some embodiments, the method of improving cholesterol levels in a subject comprises orally delivering to the subject an effective amount or concentration of one or more of gold kiwifruit powder, Lactococcus lactis, and Bacillus coagulans to achieve an improvement in cholesterol levels of the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of improving cholesterol levels in a subject, the subject's serum HDL is increased post-administration of the orally ingestible composition. In some embodiments, the subject's serum HDL is increased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving cholesterol levels in a subject, the subject's serum LDL is decreased post-administration of the orally ingestible composition. In some embodiments, the subject's serum LDL is decreased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving cholesterol levels in a subject, the subject's ratio of serum HDL to serum LDL is increased post-administration of the orally ingestible composition. In some embodiments, the subject's ratio of serum HDL to serum LDL is increased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving cholesterol levels in a subject, the subject's total serum cholesterol is decreased post-administration of the orally ingestible composition. In some embodiments, the subject's total serum cholesterol is decreased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In an embodiment, a method of alleviating IBS symptoms in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In some embodiments, the subject is a human teenager between the ages of 13 and 19.

In some embodiments, the method of alleviating IBS symptoms in a subject comprises orally delivering to the subject an effective amount or concentration of one or more of gold kiwifruit powder, Lactococcus lactis, and Bacillus coagulans to alleviate IBS symptoms of the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of alleviating IBS symptoms in a subject, the subject's abdominal pain and/or discomfort is reduced post-administration of the orally ingestible composition. In some embodiments, the subject's abdominal pain and/or discomfort is reduced by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of alleviating IBS symptoms in a subject, the subject's frequency of complete spontaneous bowel movements is increased post-administration of the orally ingestible composition. In some embodiments, the subject's frequency of complete spontaneous bowel movements is increased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In an embodiment, a method of reducing incidence of hepatic encephalopathy in a subject suffering from liver cirrhosis is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In some embodiments, the subject is a human teenager between the ages of 13 and 19.

In some embodiments, the method of reducing incidence of hepatic encephalopathy in a subject suffering from liver cirrhosis comprises orally delivering to the subject an effective amount or concentration of one or more of gold kiwifruit powder, Lactococcus lactis, and Bacillus coagulans to achieve a reduction in incidence of hepatic encephalopathy of the subject suffering from liver cirrhosis. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of reducing incidence of hepatic encephalopathy in a subject suffering from liver cirrhosis, the subject's hospital admissions are decreased in frequency post-administration of the orally ingestible composition. In some embodiments, the subject's hospital admissions are decreased in frequency by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of reducing incidence of hepatic encephalopathy in a subject suffering from liver cirrhosis, the subject's plasma ammonia levels are decreased post-administration of the orally ingestible composition. In some embodiments, the subject's plasma ammonia levels are decreased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In an embodiment, a method of improving immune response in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In some embodiments, the subject is a human teenager between the ages of 13 and 19.

In some embodiments, the method of improving immune response in a subject comprises orally delivering to the subject an effective amount or concentration of one or more of gold kiwifruit powder, Lactococcus lactis, and Bacillus coagulans to achieve an improvement in the immune response of the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of improving immune response in a subject, at least one of pDC activation, NK cell activation, and anti-viral gene expression of the subject is increased post-administration of the orally ingestible composition. In some embodiments, at least one of pDC activation, NK cell activation, and anti-viral gene expression of the subject is increased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving immune response in a subject, the subject's pathogenesis of an influenza-like illness is reduced post-administration of the orally ingestible composition. In some embodiments, the subject's pathogenesis of an influenza-like illness is reduced by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving immune response in a subject, the subject's expression of at least one of HLA-DR, VIPERIN, IFN-α, and IFN-γ is increased post-administration of the orally ingestible composition. In some embodiments, the subject's expression of at least one of HLA-DR, VIPERIN, IFN-α, and IFN-γ is increased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In an embodiment, a method of improving or alleviating one or more symptoms of an illness in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In some embodiments, the subject is a human teenager between the ages of 13 and 19.

In some embodiments, the method of improving or alleviating one or more symptoms of an illness in a subject comprises orally delivering to the subject an effective amount or concentration of one or more of gold kiwifruit powder, Lactococcus lactis, and Bacillus coagulans to achieve an improvement of one or more symptoms of an illness or to alleviate one or more symptoms of an illness of the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of improving or alleviating one or more symptoms of an illness in a subject, at least one of the subject's symptoms of sore throat, cough, feverishness, runny nose, constipation, diarrhea, abdominal pain, sneezing, muscle pain, joint pain, pain behind the eyes, head congestion, loss of appetite, fatigue, and headache are improved or alleviated post-administration of the orally ingestible composition. In some embodiments, at least one of the subject's symptoms of sore throat, cough, feverishness, runny nose, constipation, diarrhea, abdominal pain, sneezing, muscle pain, joint pain, pain behind the eyes, head congestion, loss of appetite, fatigue, and headache are improved or alleviated by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In an embodiment, a method of improving exercise or work performance in a subject is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In some embodiments, the subject is a human teenager between the ages of 13 and 19.

In some embodiments, the method of improving exercise or work performance in a subject comprises orally delivering to the subject an effective amount or concentration of one or more of gold kiwifruit powder, Lactococcus lactis, and Bacillus coagulans to achieve an improvement in exercise or work performance of the subject. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of improving exercise or work performance in a subject, the subject's fatigue, lassitude, and/or articular pain are reduced post-administration of the orally ingestible composition. In some embodiments, the subject's fatigue, lassitude, and/or articular pain are reduced by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of improving exercise or work performance in a subject, the subject's vigor, general well-being, absolute presenteeism, and/or physical condition is increased post-administration of the orally ingestible composition. In some embodiments, the subject's vigor, general well-being, absolute presenteeism, and/or physical condition is increased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In an embodiment, a method of maintaining or improving a subject's skin condition is provided. The method comprises orally administering, to the subject, an orally ingestible composition in accordance with the disclosure herein. In some embodiments, the subject is a human. In particular embodiments, the subject is a human child under the age of 18. In particular embodiments, the subject is a human child between the ages of 0 and 3. In particular embodiments, the subject is a human child between the ages of 0 and 5. In particular embodiments, the subject is a human child between the ages of 1 and 5. In particular embodiments, the subject is a human child between the ages of 6 and 10. In particular embodiments, the subject is a human child between the ages of 11 and 15. In particular embodiments, the subject is a human child between the ages of 15 and 17. In particular embodiments, the subject is a human adult. In some embodiments, the subject is a human teenager between the ages of 13 and 19.

In some embodiments, the method of maintaining or improving a subject's skin condition comprises orally delivering to the subject an effective amount or concentration of one or more of gold kiwifruit powder, Lactococcus lactis, and Bacillus coagulans to achieve maintenance or an improvement of a subject's skin condition. In some embodiments, the subject receives, via oral administration of compositions in accordance with the disclosure herein, between about 75 mg and 2800 mg of gold kiwifruit powder per day. In some embodiments, the subject receives, via oral administration of compositions in accordance with the disclosure herein, between about 5 mg and 320 mg of heat-treated Lactococcus lactis per day. In some embodiments, the subject receives, via oral administration of compositions in accordance with the disclosure herein, between about 5 mg and 800 mg of Bacillus coagulans per day. In some embodiments, the subject receives, via oral administration of compositions in accordance with the disclosure herein, between about 500 million CFUs and 80 billion CFUs Bacillus coagulans per day.

In some embodiments of the method of maintaining or improving a subject's skin condition, the subject's expression of skin homoeostasis-related genes is increased post-administration of the orally ingestible composition. In some embodiments, the subject's expression of skin homoeostasis-related genes is increased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of maintaining or improving a subject's skin condition, the subject's expression of at least one of TGFB1, BD1, BD3, S100A9, CLDN1, CLDN12, OCLN, ZO1, ZO2, and BS1 is increased post-administration of the orally ingestible composition. In some embodiments, the subject's expression of at least one of TGFB1, BD1, BD3, S100A9, CLDN1, CLDN12, OCLN, ZO1, ZO2, and BS1 is increased by at least 1%, at least 3%, at least 5%, at least 10%, at least 15%, at least 20%, at least 25%, at least 30%, at least 35%, at least 40%, at least 45%, or at least 50% post-administration of the orally ingestible composition.

In some embodiments of the method of maintaining or improving a subject's skin condition, the subject's skin dryness and/or erythema is reduced post-administration of the orally ingestible composition. In some embodiments, the subject's skin dryness and/or erythema is reduced by at least 1%, at least 3%, at least 5%, at least 10%, at least 15%, at least 20%, at least 25%, at least 30%, at least 35%, at least 40%, at least 45%, or at least 50% post-administration of the orally ingestible composition.

### III. EXAMPLES

### Example 1: Effect of Gold Kiwifruit Capsules Consumptions on Gut Microflora in Functionally Constipated Individuals

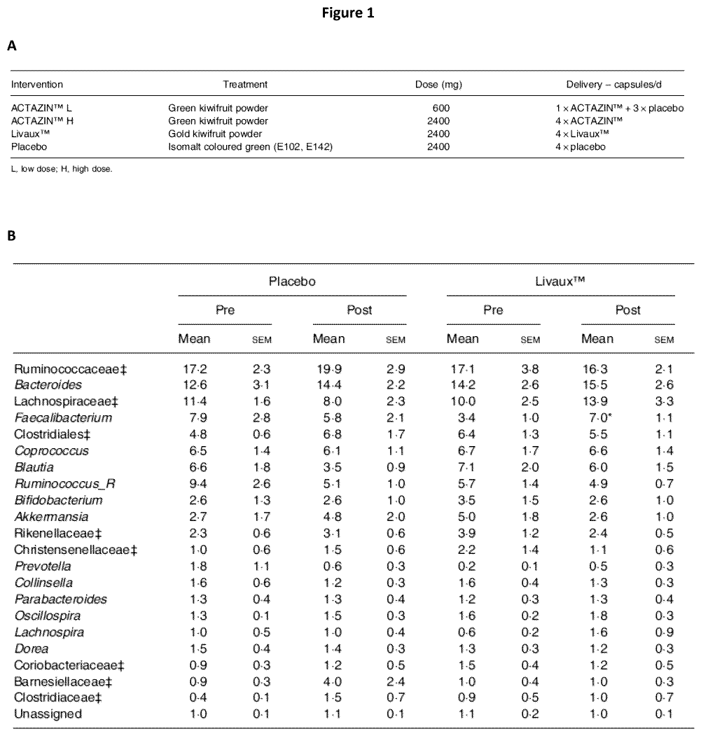

A randomized controlled human trial demonstrated that consumption of gold kiwifruit capsules (600 mg/capsule, up to 2400 mg/day for 14 days vs. isomalt placebo) significantly increased Faecalibacterium bacterial group abundance in functionally constipated subjects (see FIG. 1, from Blatchford et al., (2017) Consumption of kiwifruit capsules increases Faecalibacterium prausnitzii abundance in functionally constipated individuals: a randomized controlled human trial, Journal of Nutritional Science (2017), vol. 6, e52, page 1 of 10, herein incorporated by reference in its entirety). The study design was a randomised double-blind placebo-controlled cross-over trial with participants consuming four different interventions for 4 weeks each, with a 2-week washout between each intervention. The interventions were delivered in 4×600 mg capsules supplied by Anagenix Ltd prepared to look identical to preserve intervention blinding.

Briefly, participants consumed four different intervention combinations: placebo (isomalt coloured green) (2400 mg/d), ACTAZIN™ L (600 mg/d), ACTAZIN™ H (2400 mg/d) and Livaux™ (2400 mg/d) for 28 d each intervention, with a 14-day washout period between each treatment phase. ACTAZIN™ L (low dose, green kiwifruit) and ACTAZIN™ H (high dose, green kiwifruit) were formulated from cold-processed Actinidia chinensis var. deliciosa ‘Hayward’ green kiwifruit and Livaux™ was formulated from cold-processed Actinidia chinensis var. chinensis ‘Zesy002’ gold-fleshed kiwifruit. The placebo ingredient was isomalt (1-O-α-Dglucopyranosyl-D-mannitol). Subjects were asked to exclude high-fibre dietary supplements such as Metamucil, Benefibre and Phloe as well as maintaining their habitual dietary intakes and physical activity habits and to refrain from eating fresh kiwifruit for the study period. At the beginning and end of each 4-week intervention period, participants were asked to provide a faecal sample. The washout period of 2 weeks was chosen to allow sufficient time to return bowel habits to baseline for the parameters measured (microbial ecology, microbial metabolites and SmartPill® pH measurements).

This study investigated the impact of ACTAZIN™ green (2400 and 600 mg) and Livaux™ (2400 mg) gold kiwifruit supplements on faecal microbial composition and metabolites in healthy and functionally constipated (FC) participants. The participants were recruited into the healthy group (n 20; one of whom did not complete the study) and the FC group (n 9), each of whom consumed all the treatments and a placebo (isomalt) for 4 weeks in a randomized cross-over design interspersed with 2-week washout periods. Modification of faecal microbiota composition and metabolism was determined by 16S rRNA gene sequencing and GC, and colonic pH was calculated using SmartPill® wireless motility capsules. A total of thirty-two taxa were measured at greater than 1% abundance in at least one sample, ten of which differed significantly between the baseline healthy and FC groups. Specifically, Bacteroidales and Roseburia spp. Were significantly more abundant (P<0-05) in the healthy group, and taxa including Ruminococcaceae, Dorea spp. And Akkermansia spp. Were significantly more abundant (P<0·05) in the FC group. In the FC group, Faecalibacterium prausnitzii abundance significantly increased (P=0·024) from 3.4 to 7.0% following Livaux™ supplementation, with eight of the nine participants showing a net increase. Lower proportions of F. prausnitzii are often associated with gastrointestinal disorders. The discovery that Livaux™ supplementation increased F. prausnitzii abundance offers a potential strategy for improving gut microbiota composition, as F. prausnitzii is a butyrate producer and has also been shown to exert anti-inflammatory effects in many studies.

### Example 2: Effect of Gold Kiwifruit on Bowel Movements

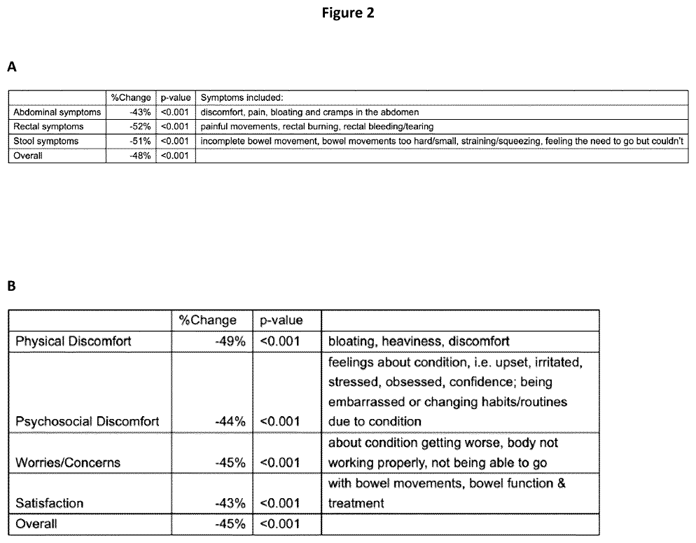

Participants were administered Livaux in a placebo-controlled study. Participants treated with Livaux saw a 162% increase in complete bowel movements as compared to a 35% increase seen in the placebo group. The group administered Livaux also saw a statistically significant decrease in abdominal symptoms (e.g. discomfort, pain, bloating and cramps), rectal symptoms (e.g. painful movements, rectal burning, rectal bleeding/tearing), and stool symptoms (e.g. incomplete bowel movement, hard/small bowel movements, straining, squeezing) (FIG. 2A). The Livaux treated group saw a reduction in psychosocial discomfort and increase in satisfaction (FIG. 2B).

### Example 3: Effect of Gold Kiwifruit on Immune Response

A crossover study investigated the effects of gold kiwifruit on the duration and severity of cold and flu symptoms and the subjects' immune response. Older adult subjects were divided into two groups. One group had an intake period followed by a non-intake period; the other group had a non-intake period followed by an intake period. During the intake period, subjects consumed freeze-dried gold kiwifruit. During the non-intake period, subjects consumed freeze dried banana. Compared to gold kiwifruit, banana provides similar energy content but lower nutritional value. The older adult subjects experienced fewer symptoms of sore throat and head congestion when they consumed kiwifruit compared to banana. The older adult subjects experienced a sore throat for a mean of 5.4 days when they ate banana; when they ate the kiwifruit, the older adult subjects experienced a mean of only 2.0 days (p=0.02). Head congestion symptoms were reduced from 4.7 days when consuming banana to 0.9 days when consuming kiwifruit (p=0.03). The older adult subjects also experienced less severe head congestion when consuming kiwifruit. The older adult subjects experienced head congestion at an average severity score of 6.72 out of 10 when consuming banana but an average score of 1.3 out of 10 when consuming kiwifruit (p=0.01).

The immune response in children were also investigated. The children were divided into two groups. One group consumed two servings of gold kiwifruit five days a week for four weeks; the other group consumed two servings of banana five days a week for four weeks. The children's parents recorded the children's cold and flu symptoms. The children who consumed the kiwifruit experienced ad 0.55 odds ratio of having a cold or flu-like illness compared to the children who consumed banana. The children who consumed kiwifruit also experienced less symptoms including having a better appetite (p=0.01), not feeling as unwell (p=0.04), having more energy (p=0.01), crying less (p=0.04), less severe headaches (p=0.02), and less severe sore throats (p=0.04) compared to the children who consumed banana.

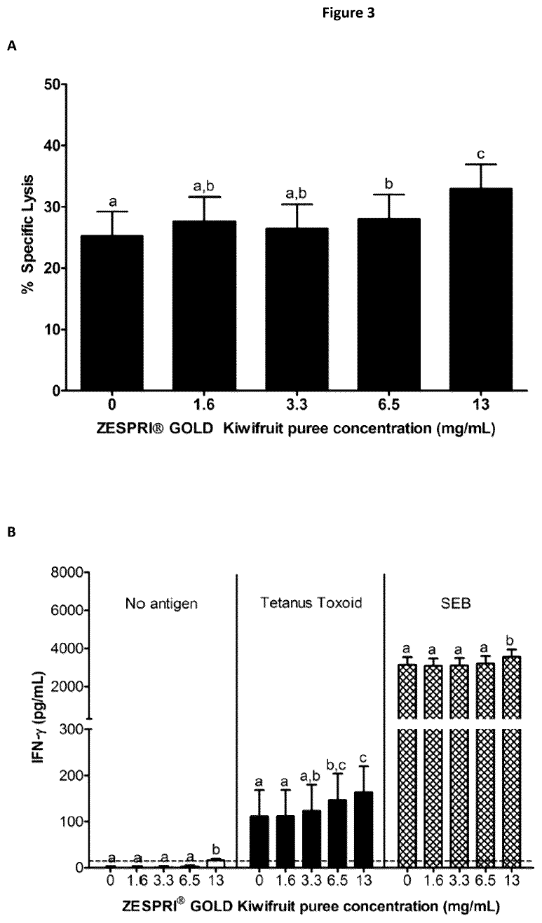

The effect of gold kiwifruit on natural killer cell activity and recal interferon 7 (IFN γ) response was measured by incubating blood samples or white blood cell fractions taken from human volunteers. The blood cells were incubated with four different concentrations of gold kiwifruit puree: 1.6 mg/mL, 3.3 mg/mL, 65 mg/mL, and 13 mg/mL. Natural killer cell activity was measured by the ability of peripheral blood mononuclear cells to lyse K562 natural killer target cells. IFN-γ production after 24 hours in the absence or presence of tetanus toxoid was measured by ELISA. Natural killer cell activity was significantly increased (FIG. 3A). IFN-γ secretion increased as the concentration of gold kiwifruit increased (FIG. 3B). See Skinner, M. A., “Wellness Foods Based on the Health Benefits of Fruit: Gold Kiwifruit for Immune Support and Reducing Symptoms of Colds and Influenza,” Journal of Food and Drug Analysis, Vol. 20, Suppl. 1, 2012, pp. 261-264, herein incorporated by reference in its entirety.

### Example 4: Effect of Bacillus coagulans Unique IS2 in Constipation

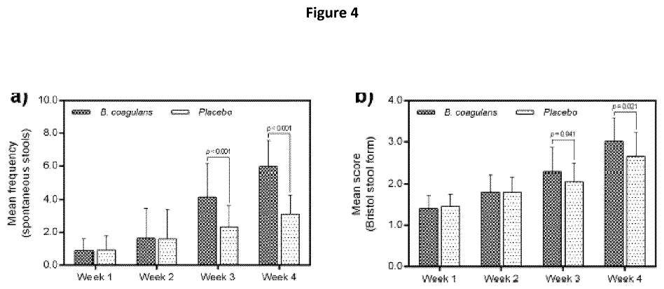

Functional constipation has a high prevalence in both adults and children affecting quality of life. Evidence suggests that probiotics can reduce the symptoms of constipation. As the effects of probiotics are strain specific, the efficacy of Bacillus coagulans Unique IS2 in the treatment of functional constipation in adults was evaluated. Subjects (n=100) diagnosed with functional constipation were supplemented with either B. coagulans Unique IS2 (2 billion CFU) or placebo capsules once daily for 4 weeks. Subjects were evaluated for treatment success (defined as three or more spontaneous stools per week), stool consistency, difficulty of defecation, defecation, and abdominal pain. By the end of the fourth week, there was a statistically significant (p<0.001) increase in number of bowel movements in the probiotic treated group as compared to placebo (FIG. 4). Ninety eight percent of subjects in the probiotic group achieved normal stool consistency as compared to placebo (74%). Moreover, there was relief from symptoms of incomplete evacuation, painful defecation, and abdominal pain associated with constipation in probiotic treated group as compared to placebo. In conclusion, B. coagulans Unique IS2 significantly decreased the symptoms of constipation indicating effectiveness of the strain in the treatment of constipation.

### Example 5: Effect of Bacillus coagulans Unique IS-2 in the Treatment of Patients with Bacterial Vaginosis

Control group was given standard vaginosis treatment alone (Ofloxacin-Ornidazole 200-500 mg/capsule/day for 5 days along with co-kimaxazol vaginal peccaries for 3 days), as the test group took standard vaginosis treatment plus 2 capsules of B. coagulans (10⁹ CFU/capsule) twice a day for 90 days, from Sudha et al., 2011, Clinical Study of Bacillus coagulans Unique IS-2 (ATCC PTA-11748) in the Treatment of Patients with Bacterial Vaginosis, Indian J Microbiol (July-September 2012) 52(3):396-399 397, herein incorporated by reference in its entirety.

Forty women reported at out-patient department in Government Medical College and Hospital Aurangabad, India, with BV were examined. The study was approved by the Institutional Ethics Committee (ref no: ICE/GMCA/114/2008) and all subjects gave written informed consent before any research activity was initiated. The inclusion criteria for the subjects was based on presence of the following symptoms such as, white discharge, positive for whiff test, vaginal pH greater than 4.7 (normal pH between 3.8 and 4.5), along with other symptoms like, burning micturition, itching, soreness and redness at vulva.

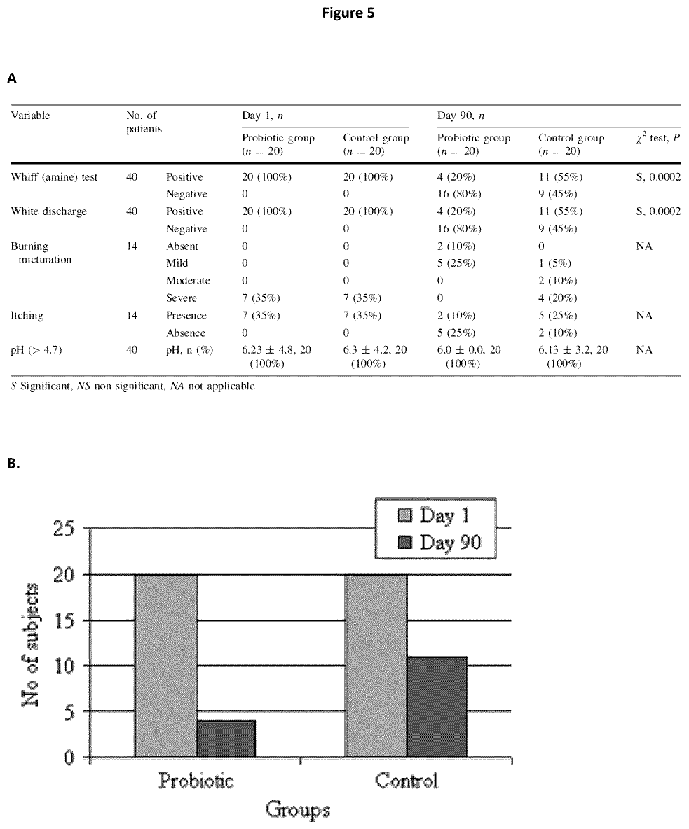

Participants fulfilling the entry criteria were divided equally (control 20 and probiotic 20). The following observation are noted for age (control group, 33±3 years and probiotic group, 32.5±3), history of previous vaginosis (control group, 75% or 15/20 and probiotic group, 75% or 15/20) and severity of current vaginosis infection (burning micturition and itching, 35% in each group). Study subjects were not blinded to the treatment they received. The control group patients were given standard vaginosis treatment alone (Ofloxacin-Ornidazole with a strength of 200-500 mg per capsule/day for 5 days along with co-kimaxazol vaginal peccaries for 3 days). The probiotic group patients took standard vaginosis treatment plus two capsules of B. coagulans Unique IS-2 (1×9 109 CFU/capsule) twice a day before meals for 90 days. At the end of the treatment the 80% of probiotic group subjects showed significant positive response as revealed by reduction of vaginosis symptoms compared to the control group which exhibited reduction in 45% subjects only (FIGS. 5A and 5B). Thus, the results of present study indicate that strain Bacillus coagulans Unique IS-2 can provide benefits to women being treated with antibiotics to cure an infectious condition.

### Example 6: Effect of Bacillus coagulans on Plaque Induced Gingivitis

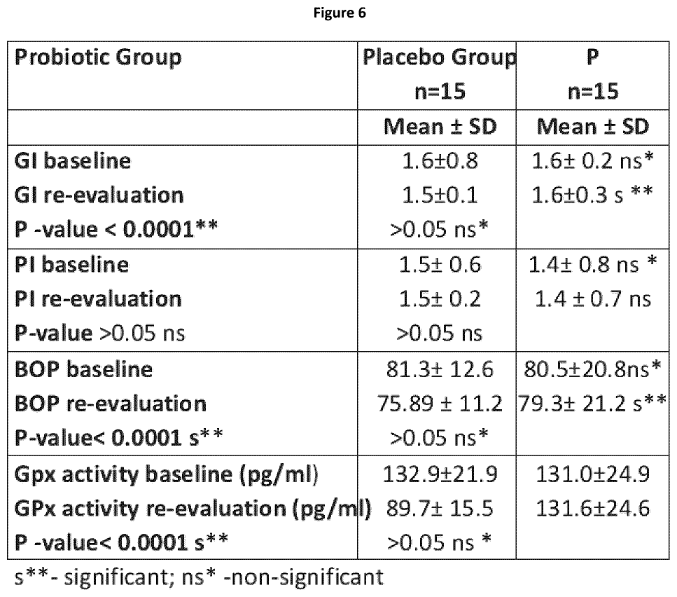

Thirty subjects with plaque induced gingivitis were enrolled in the study. At baseline, gingival index (GI) and plaque index (PI) were assessed. Saliva samples were collected for glutathione peroxidase (GPx) activity analysis and to determine load of lactobacilli. Subsequently, participants were randomly provided with chewable tablets to be consumed 3 times daily for 3 months containing 100 million CFU/tablet of B. coagulans or without B. coagulans (placebo). After 3 months, recording of GI, PI, and saliva sampling were repeated. At baseline, mean GI, and mean PI did not differ significantly between groups. At re-evaluation, mean GI, and bleeding on probing of the probiotic group were both significantly lower (p<0.0001) than in the placebo group. Mean PI level did not differ significantly between the groups. In the probiotic group, mean GPx activity was significantly lower (p<0.02) than in the placebo group at re-evaluation (see FIG. 6), from Jagadeesh et al., Clinical effect of pro-biotic containing Bacillus coagulans on plaque induced gingivitis: a randomised clinical pilot study, NUJHS Vol. 7, No. 3, September 2017, herein incorporated by reference in its entirety.

### Example 7: Effect of Bacillus coagulans Strain Unique IS-2 in the Treatment of Patients with Acute Diarrhea

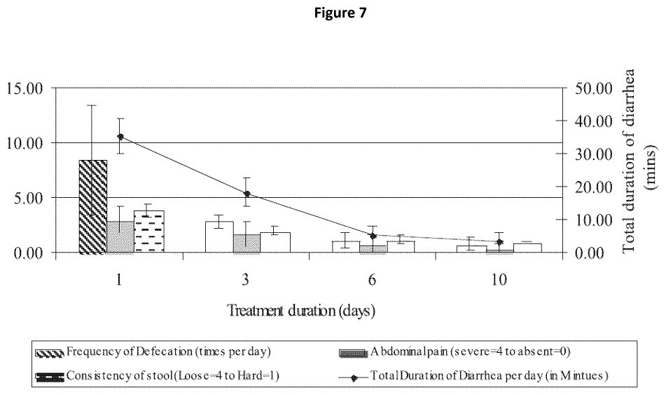

The objective of this study was to evaluate the efficacy and safety of Bacillus coagulans strain Unique IS-2 in the treatment of patients with acute diarrhea. To this end, a total of 28 patients with acute diarrhea were included in a prospective, phase II clinical study upon obtaining consent and ethical committee approval. The trial was performed on patients of both sexes between 18 to 55 years of age and having grade 3 loose motions in last 24 hours for less than 7 Days. All patients were assigned to receive capsule (containing 2 billion CFU of Bacillus coagulans strain Unique IS-2) two times for a duration of 10 days. Efficacy was evaluated by assessment of duration of diarrhea (in minutes) and frequency of defecation (times per day), abdominal pain (3=severe; 2=moderate; 1=mild; 0=absent) and consistency of stool (1=normal, 2=loose, 3=semi liquid, and 4=liquid). Safety aspects of capsule were evaluated by assessment of incidence, type of adverse events, physical examination, and clinical laboratory test values (CBC, SGPT, serum creatinine, stool routine and microscopy). Concomitant medications including rescue medications were monitored throughout the study. Efficacy assessment for duration of diarrhea, frequency of defecation, abdominal pain, consistency of stool and fever was done on 1, 3, 6 and 10 days. Mean values for duration of diarrhea decreased from 35.60±5.46 to 3.52±2.69 min per day; frequency of defecation was decreased from 7.96±3.89 to 0.76±0.60 times per day; abdominal pain decreased from 3.16±0.99 to 0.36±0.49; and consistency of stool improved from 3.84±0.55 to 1.00±0.00. No significant change in safety parameters was observed during treatment. This trial demonstrates that utilization of B. coagulans Unique IS-2 strain is efficient and safe to treat the patients with acute diarrhea (see FIG. 7), from Sudha and Bhonagri, 2011, Efficacy Of Bacillus Coagulans Strain Unique Is-2 In The Treatment Of Patients With Acute Diarrhea, International Journal of Probiotics and Prebiotics Vol. 7, No. 1, 2012, herein incorporated by reference in its entirety.

### Example 8: Effect of Bacillus coagulans Unique IS-2 with Lactulose on Functional Constipation in Adults

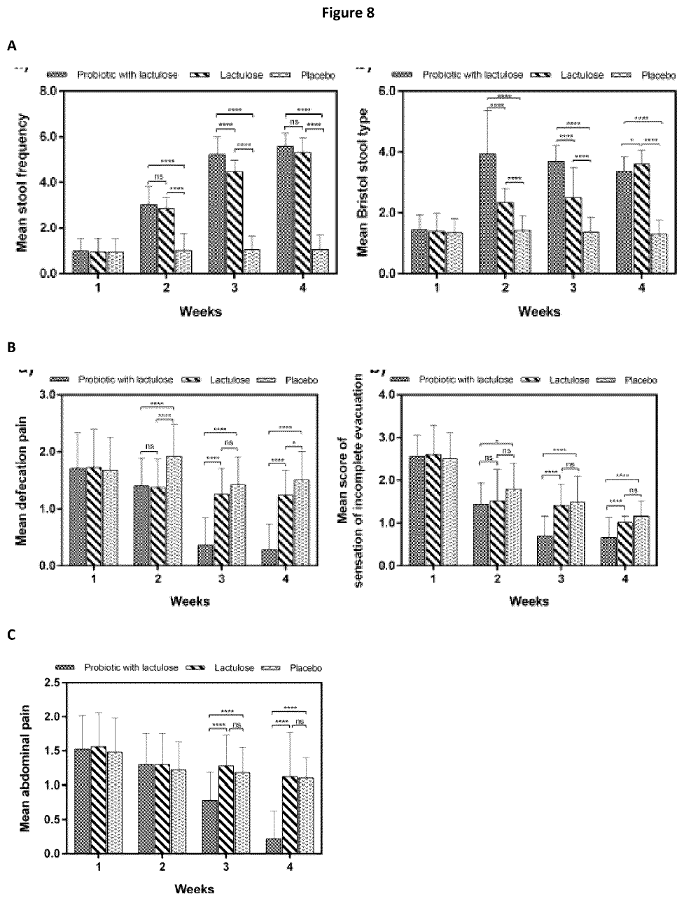

This double-blind randomized study evaluated the efficacy of a combination of Bacillus coagulans Unique IS-2 and lactulose in the treatment of functional constipation in adults. In it, 150 participants diagnosed with functional constipation (Rome III criteria) were randomised (1:1:1) and supplemented daily with 15 mL suspension of probiotic (B. coagulans Unique IS-2, 2×109 spores) with lactulose (10 g) (group 1) or lactulose (10 g) (group 2) or placebo (water) (group 3) for 4 weeks. The primary (stool frequency) and secondary outcome measures (stool consistency, sensation of incomplete evacuation, defecation- and abdominal pain) were recorded weekly for up to 4 weeks. Bacillus coagulans Unique IS-2 with lactulose showed significant changes in stool frequency as compared to lactulose treatment alone; however, at the end of the trial, it was found insignificant due to the gradual increase of stool frequency score of lactulose treatment. The changes observed in stool consistency were early (2nd week) and remained consistent up to end of the trial. The significant reduction of sensation of incomplete evacuation, defecation pain, and abdominal pain correlated with the strain's ability to produce short-chain fatty acids. No adverse events were observed in any of the groups, and all the vital parameters were normal during the course of the study. Overall, results indicated that the addition of B. coagulans Unique IS-2 to lactulose reduced time required to relieve constipation as compared to lactulose alone. In conclusion, B. coagulans Unique IS-2 with lactulose is more effective than lactulose alone to relieve symptoms of constipation in a shorter period. (see FIG. 8), from Venkataraman et al., Effect of Bacillus coagulans Unique IS2 with Lactulose on Functional Constipation in Adults: a Double-Blind Placebo Controlled Study, Probiotics and Antimicrobial Proteins https://doi.org/10.1007/si2602-021-09855-8, herein incorporated by reference in its entirety.

### Example 9: Effect of Bacillus coagulans on Protein Absorption

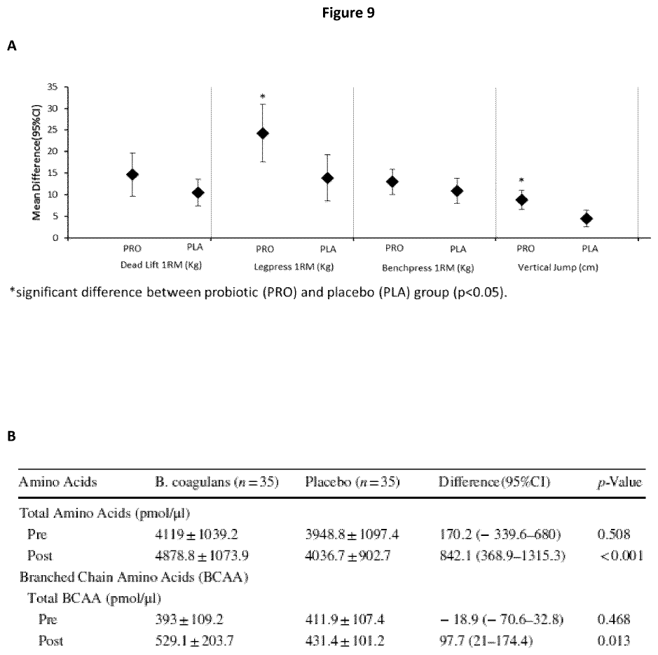

In this double blind, placebo-control trial, resistance-trained males (21.08±2.84 years) were randomized to consume, either 20 g of whey protein powder {80% whey protein concentrate (WPC80), amounting to 15.4 g protein} with 2 billion CFU Bacillus coagulans Unique IS-2 (supplemental group) or 20 g of whey protein powder and lactose instead of Bacillus coagulans (placebo group) once daily for 60 days with a controlled resistance exercise protocol. The whey protein concentrate (WPC-80) given to both groups had a lactose content of 6.8%. Plasma-free amino acids (PFAAs) were determined at baseline, at 30 and 60 days of supplementation. Muscle strength, hypertrophy, VO2 max, and body composition, and other biochemical parameters were assessed at baseline and end line. Probiotic effect was shown on exercise performance as evidenced by an increase in one RM of leg press and vertical jump power by +16.61% (p=0.024) and +7.86% (p=0.007), respectively (FIG. 9A). A positive effect of probiotic Bacillus coagulans Unique IS-2 supplementation was observed on protein absorption as evidenced by an increase in total PFAA by +16.1% (p=0.004) (FIG. 9B, partial results shown). Branched chain amino acids (BCAA) comprising isoleucine (p=0.016), leucine (p=0.001), and valine (p=0.002) were increased by +33.1% in ITT analysis as compared to placebo after 60 days. At 30 days an increase in isoleucine by +35% (p=0.113), leucine by +43% (p=0.032), and valine by +32% (p=0.017) was observed in ITT analysis. In conclusion, significantly increased absorption of BCAA with supplementation of B. coagulans Unique IS-2 along with whey protein and improvement in leg press and vertical jump power was noted indicating the positive effect of the probiotic on muscle power in the lower body. See Tarik et al., The effect of Bacillus coagulans Unique IS-2 supplementation on plasma amino acid levels and muscle strength in resistance trained males consuming whey protein: a double-blind, placebo-controlled study, European Journal of Nutrition, https://doi.org/10.1007/s00394-022-02844-9, herein incorporated by reference in its entirety.

### Example 10: Effect of B. coagulans Supplementation on Serum Cholesterol

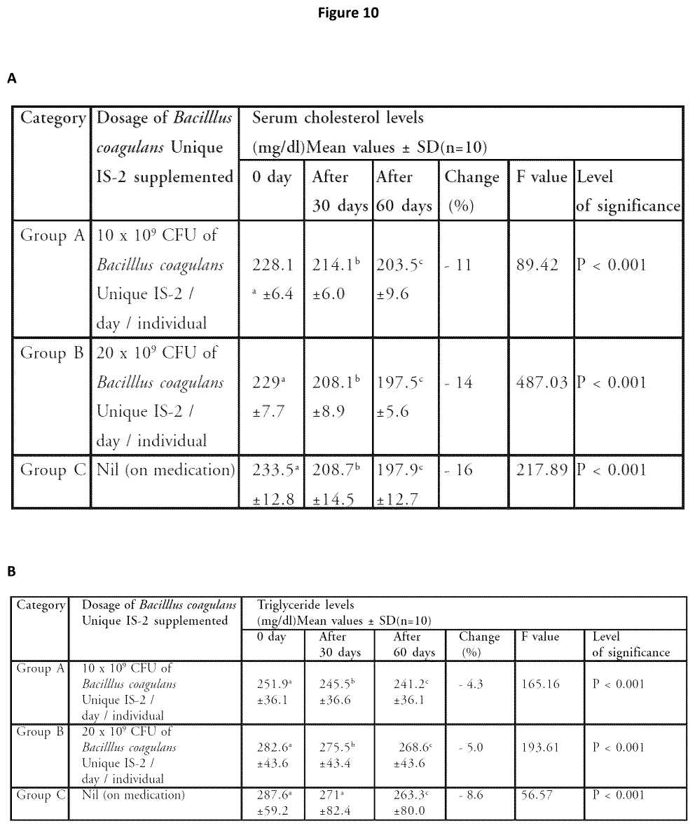

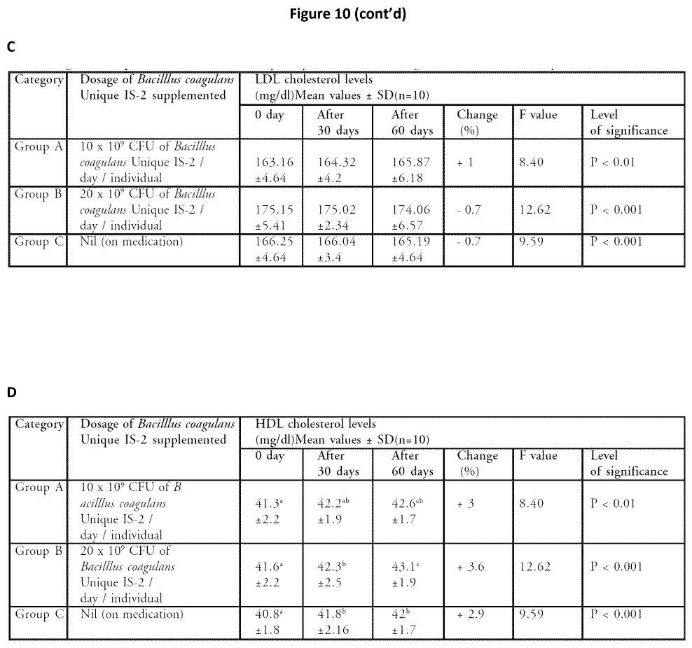

The objective of this study was to evaluate the effect of dietary supplementation of capsules containing Bacillus coagulans Unique IS-2, on serum lipids for 60 days. Thirty hyperlipidemic (having serum cholesterol levels of more than 200 mg/dl) subjects were divided into 3 groups (n=10). Two group subjects were allotted to receive a daily dose of two capsules of probiotic Bacillus coagulans Unique IS2 (10×10⁹ CFU/capsule (Group A) and 20×10⁹ CFU/capsule (Group B)) and third group subjects received standard medication. Serum lipid profiles were taken at 0 days, 30 days, and 60 days of the study period. At the end of study there were slight reductions in total cholesterol (FIG. 10A), triglycerides (FIG. 10B) and LDL (FIG. 10C), and an increase in HDL cholesterol levels (FIG. 10D). These data suggest that the strain Bacillus coagulans IS-2 has the potential of aiding control of the serum cholesterol in humans suffering with hyperlipidemia. Taken from Sudha et al, Effect Of Supplementation Of Probiotic Bacillus Coagulans Unique Is-2 On Hypercholesterolemia Subjects: A Clinical Study, International Journal of Probiotics and Prebiotics Vol. 6, No. 2, 2011, herein incorporated by reference in its entirety.

### Example 11: Effect of Bacillus coagulans Supplementation on Dental Caries in Children

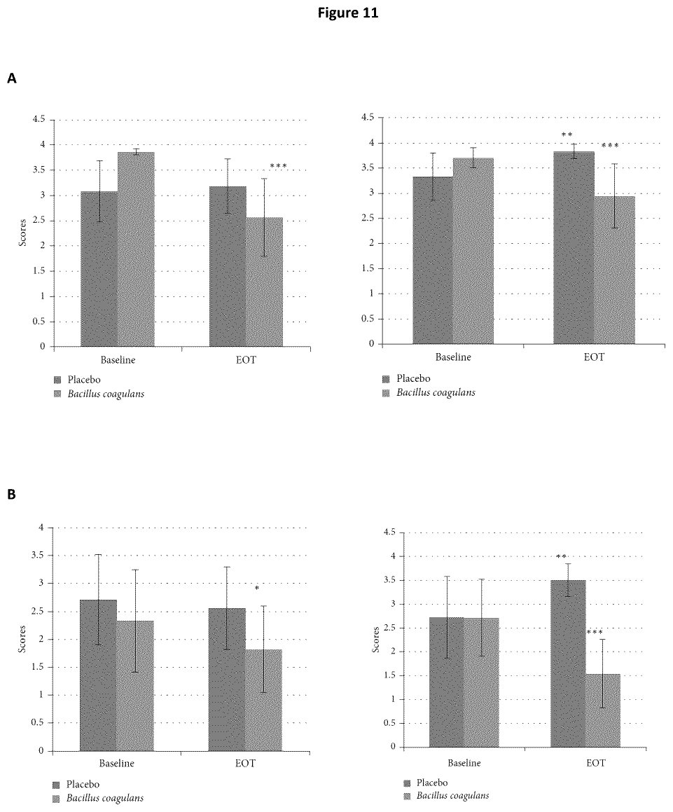

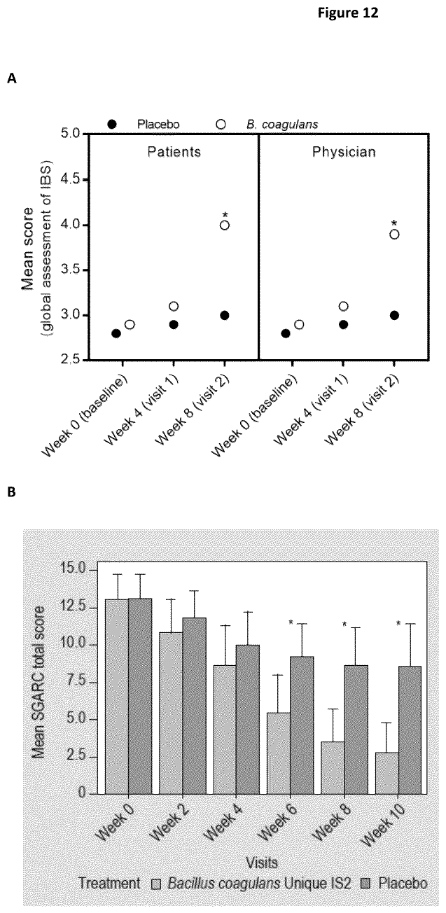

Dental caries or tooth decay is caused by demineralization of the tooth enamel, leading to the breakdown of the enamel causing cavities to be formed. Demineralization of the tooth happens because of the acid secreted by bacteria like mutans streptococci and lactobacilli. Probiotic usage may prevent the overgrowth of these pathogenic microbes, thereby reducing caries activity. In this double-blind, randomized, placebo-controlled study, children with ages ranging from 5 to 15 years were divided into two groups, the probiotic and placebo groups. Chewable tablets with and without probiotic Bacillus coagulans Unique IS-2 were administered for two weeks. Stimulated saliva samples and plaque were collected at baseline and at the end of 14 days to measure the pH, mutans streptococci, and lactobacilli count of saliva and plaque using chairside kits. A statistically significant reduction in mutans streptococci and lactobacilli counts of both saliva and plaque samples was observed in the B. coagulans Unique IS-2 treated group after 14 days of administration compared to the baseline and placebo group (using paired t-test) (FIG. 11). Probiotic Bacillus coagulans Unique IS-2 (2 billion cfu) chewable tablet is effective in reduction and inhibition of caries causing mutans streptococci and lactobacilli levels in saliva and plaque in children. See Sudha et al., Evaluation of the Effect of Probiotic Bacillus coagulans Unique IS-2 on Mutans Streptococci and Lactobacilli Levels in Saliva and Plaque: A Double-Blind, Randomized, Placebo-Controlled Study in Children, International Journal of Dentistry Volume 2020, Article ID 8891708, 8 pages, herein incorporated by reference in its entirety.

### Example 12: Effect of Bacillus coagulans Supplementation on Irritable Bowel Syndrome in Adults and in Children

In this study the efficacy of B. coagulans Unique IS-2 in the management of IBS symptoms in adults was investigated. Patients (n=153) fulfilling Rome III criteria were provided placebo capsules for a 2 weeks run-in period. Only patients satisfying compliance criteria (n=136) were randomized (double blind) to receive either B. coagulans Unique IS-2 (2 billion CFU) or placebo capsules daily for 8 weeks. Reduction of abdominal discomfort/pain intensity and increase in complete spontaneous bowel movements were analyzed as primary end points. Other clinical symptoms of IBS and serum cytokines were also evaluated. B. coagulans Unique IS-2 showed significant improvement in primary and secondary endpoints, as compared to placebo. Haematology of both the arms remained normal. No significant changes in pro (IL-6, IL-12, TNF-α, INF-γ) and anti-inflammatory cytokine (IL-10) levels were detected at the end of B. coagulans treatment (8 weeks) as compared to placebo. B. coagulans was well tolerated with no severe adverse events to report. Overall, the results demonstrate that B. coagulans Unique IS-2 is efficacious in the management of IBS symptoms in adults (18-60 years) (FIG. 12A). Taken from Madempudi et al., Randomized clinical trial: the effect of probiotic Bacillus coagulans Unique IS-2 vs. placebo on the symptoms management of irritable bowel syndrome in adults, Scientific Reports (2019) 9:12210, herein incorporated by reference in its entirety.

The efficacy of the probiotic strain, Bacillus coagulans Unique IS-2 in the treatment of Irritable Bowel Syndrome (IBS) was evaluated in children. A total of 141 children of either sex in the age group 4-12 years, diagnosed with IBS according to the Rome III criteria, participated in the double-blind randomised controlled trial. Children received either B. coagulans Unique IS-2 chewable tablets (2×10⁹ CFU, using dextrose as an excipient) or placebo (dextrose excipient only) once daily for eight weeks followed by a two week follow-up period. Reduction in pain intensity as well as other symptoms associated with Irritable Bowel Syndrome like abdominal discomfort, bloating, distension, sense of incomplete evacuation, straining at stool, urgency of bowel movement, passage of gas and mucus, and bowel habit satisfaction were assessed. B. coagulans Unique IS-2 treated group showed a greater reduction in pain scores as evaluated by a weekly pain intensity scale. There was a significant reduction (P<0.0001) in pain intensity in the probiotic treated group (7.6±0.98) as compared to the placebo group (4.2±1.41) by the end of the treatment period (8 weeks). There was also a significant improvement in stool consistency as well as reduction in abdominal discomfort, bloating, staining, urgency, incomplete evacuation and passage of gas. Bowel habit satisfaction and global assessment of relief was also observed in the B. coagulans Unique IS-2 treated group as compared to the placebo group. This study demonstrates the efficacy of B. coagulans Unique IS-2 in reducing the symptoms of Irritable Bowel Syndrome in children in the age group of 4-12 years (FIG. 12B). Talen from Sudha et al., Efficacy of Bacillus coagulans Unique IS-2 in treatment of irritable bowel syndrome in children: a double blind, randomised placebo controlled study, Beneficial Microbes, 2016; in press, herein incorporated by reference in its entirety.

### Examples 13: Effect of Lactobacilli Sporogenes (B. Coagulans) on Liver Cirrhosis

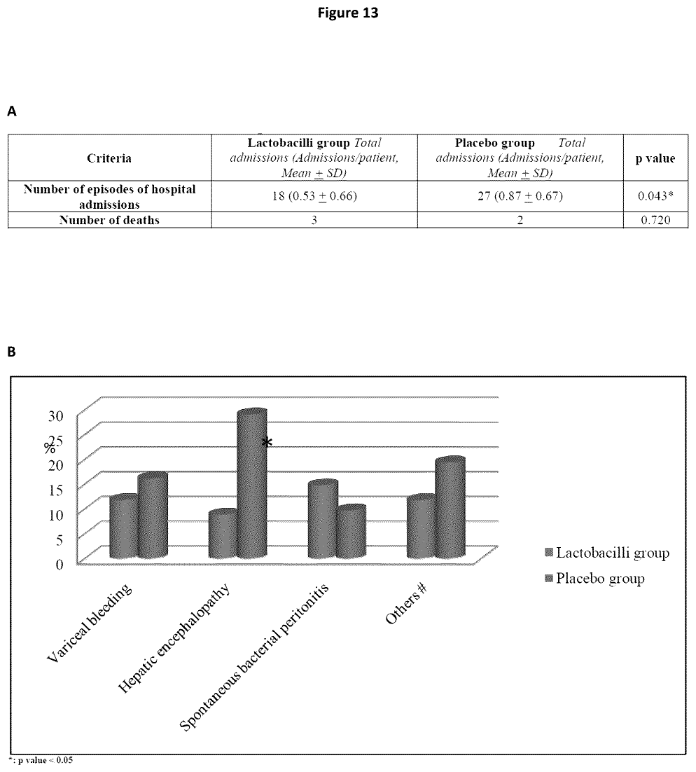

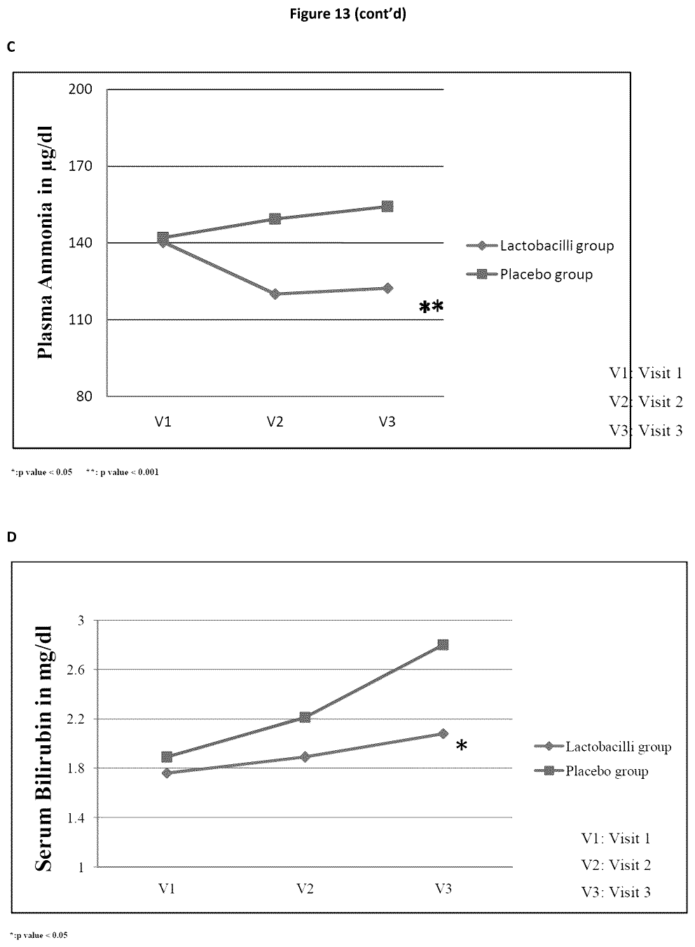

A randomized, double-blind, placebo-controlled study investigated the effect of lactobacilli (B. coagulans) on liver cirrhosis. Patients with liver cirrhosis were divided into two groups. The treated group (n=34) consumed two capsules containing 2 billion CFUs lactobacilli twice daily for six months. The placebo group (n=31) consumed placebo capsules twice daily for six months. The treated group experienced fewer episodes of hospital admission than the placebo group (FIG. 13A, p=0.043). The lactobacilli group experienced fewer incidences of complications of cirrhosis like hepatic encephalopathy (FIG. 13B, p<0.05). The placebo group experienced increased plasma ammonia levels during the treatment period, but the treated group saw overall decreased plasma ammonia levels (FIG. 13C, p<0.001). During the treatment period, the group which consumed Lactobacilli experienced a significantly smaller increase in serum bilirubin levels compared to the placebo group (FIG. 13D, p<0.05). See Pawar, R. R. et al., “Study of Effects of Probiotic Lactobacilli in Preventing Major Complications in Patients of Liver Cirrhosis,” International Journal of Research in Pharmaceutical and Biomedical Sciences, vol 3, no. 1, (2012) pp. 206-211, herein incorporated by reference in its entirety.

### Example 14: Efficacy of Bacillus coagulans on Small Intestinal Bacterial Overgrowth

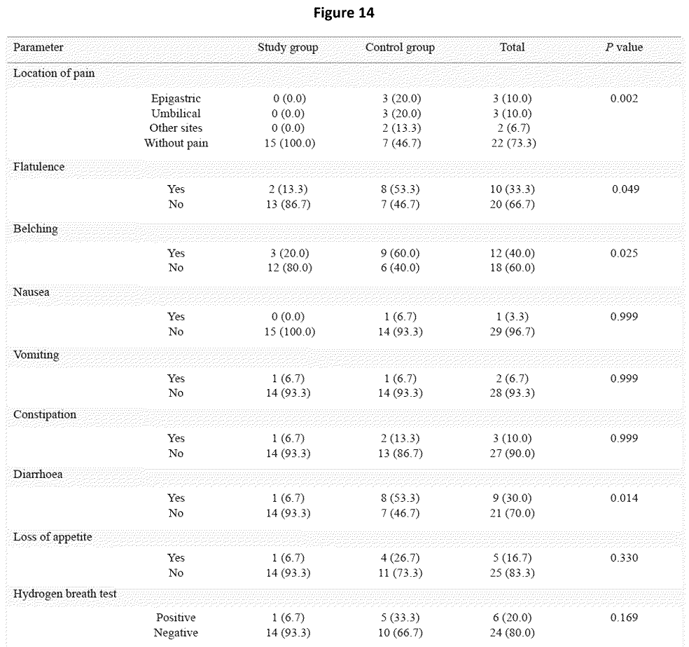

A randomized, double-blind, placebo-controlled study investigated the effects of Bacillus coagulans on GI symptoms caused by small intestinal bacterial overgrowth (SIBO). Patients experiencing chronic stomach pain or changes in defecation without a diagnosis other than SIBO were screened for participation in the study. Patients meeting the criteria underwent a hydrogen breath test (HBT); subjects with thirty consecutive days with a positive HBT result were included in the study. The subjects were randomly assigned to two groups: a treated group (n=15) and control group (n=15). The treated group underwent three weeks of broad-spectrum antibiotics, followed by six months of a monthly regimen of 15 days of twice a day minocycline antiobiotic therapy in the first half of the month and 15 days of twice a day lactol probiotic containing Bacillus coagulans and fructo-oligosaccharides in the second half of the month. The control group underwent three weeks of broad-spectrum antibiotics followed by six months of a monthly regimen of 15 days of twice a day minocycline antibiotic therapy in the first half of the month. The control group did not take anything in the second half of each month during the 6 month regimen period. The subjects recorded their GI symptoms and underwent a HBT after the treatment period. At the end of the treatment period, the treated group saw significant reduction in pain (p=0.002), flatulence (p=0.049), belching (p=0.025), and diarrhea (p=0.014) (FIG. 14). See Khalighi, A. R. et al, “Evaluating the efficacy of probiotic on treatment in patients with small intestinal bacterial overgrowth (SIBO)—A pilot study,” Indian J. Med. Res. 140 (2014) pp. 604-608, herein incorporated by reference in its entirety.

### Example 15: Anti-Inflammatory Activity of Lactobacillus sporogenes (Bacillus coagulans)

A study investigated the effect of Bacillus coagulans and Bifidobacterium bifidum on carrageenan-induced paw edema in Wistar rats. The rats were divided into five groups each containing six rats: Group A was a vehicle control only receiving 500 μL of distilled water; Group B was a carrageenan control group only receiving 500 μL of distilled water; Group C received 108 CFUs Bacillus coagulans suspended in 500 μL of distilled water; Group D received 108 CFUs Bifidobacterium bifidum suspended in 500 μL of distilled water oral treatment; and Group E was treated with 150 mg/kg body weight of diclofenac sodium as a reference standard anti-inflammatory drug. Groups C and D were further divided into 2 groups with three rats in each: Group C1 received 108 CFUs of non-encapsulated Bacillus coagulans; Group C2 received 108 CFUs of Bacillus coagulans as probiotic beads; Group D1 received 108 CFUs of non-encapsulated Bifidobacterium bifidum; and Group D2 received 108 CFUs of Bifidobacterium bifidum as probiotic beads.

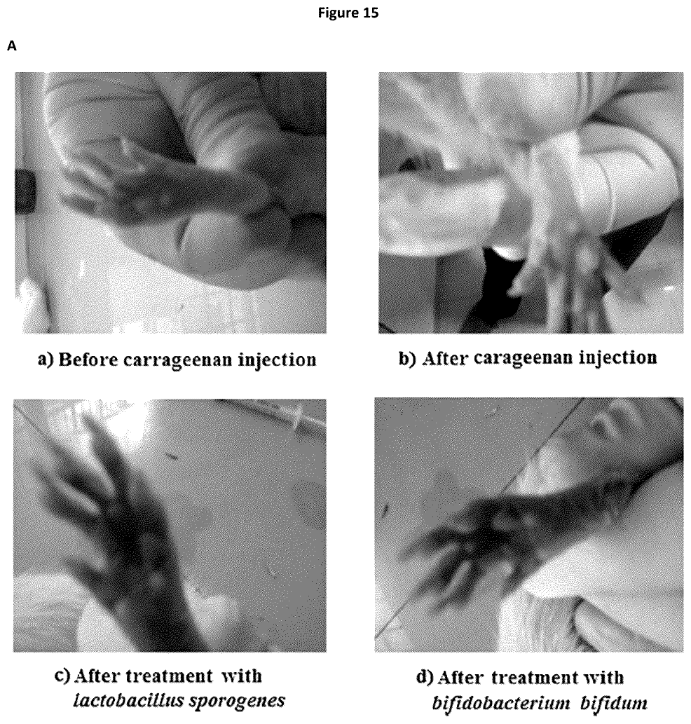

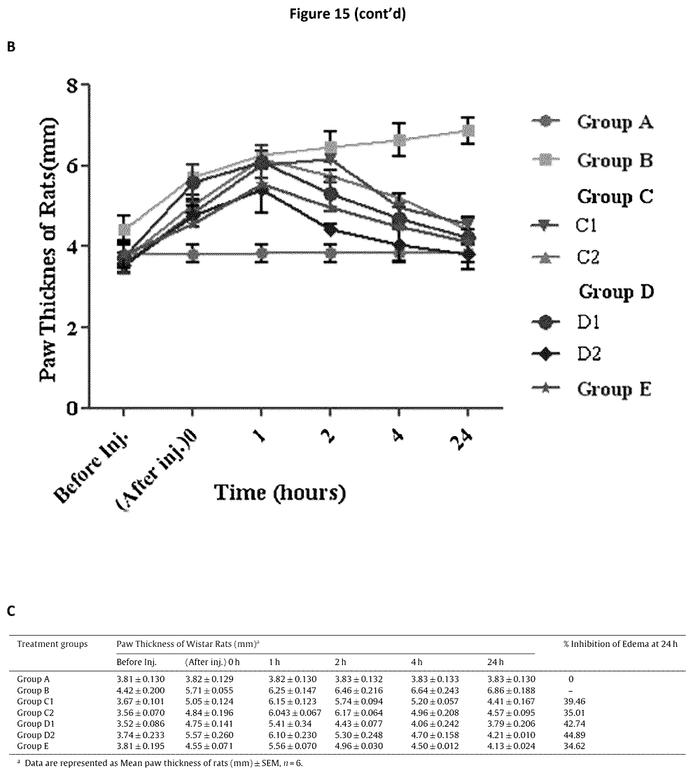

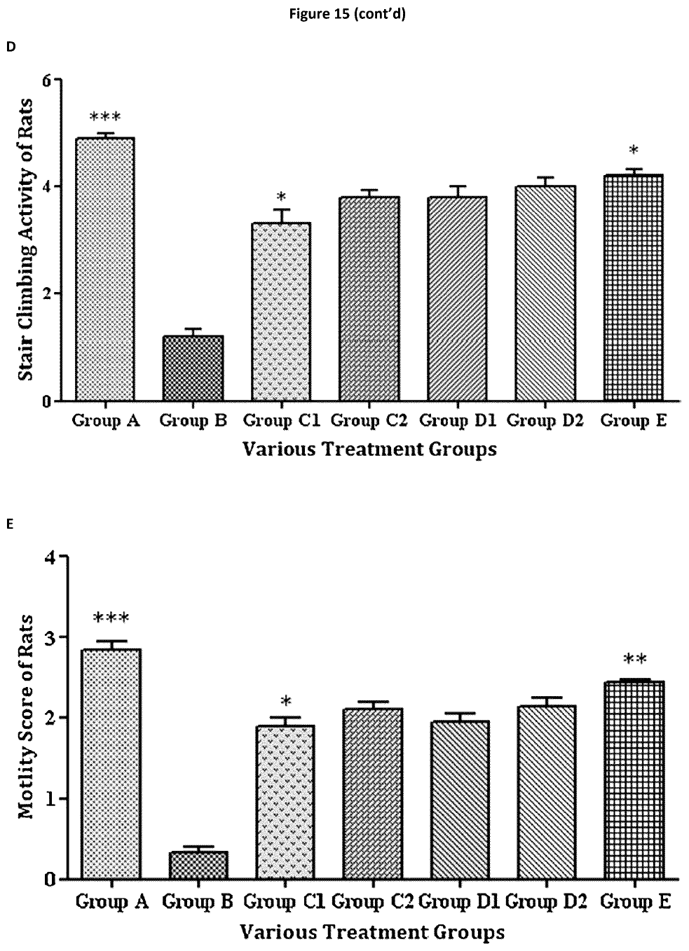

All groups except Group A were administered 100 μL of 1% carrageenan in distilled water (FIG. 15A). Rats in all groups were administered a single dose of their respective treatments. The rats' paw thickness was measured 30 min prior to injection with carrageenan and after the injection at 0, 1, 2, 3, 4, and 24 hours after injection (FIGS. 15B and 15C). The rats treated with B. coagulans (Groups C1 and C2) experienced 35-40% inhibition of edema at 24 hours. The rats treated with B. bifidum (Groups D1 and D2) experienced between 42-45% inhibition of edema at 24 hours. The rats administered diclofenac sodium (Group E) experiences about 34% inhibition of edema at 24 hours. The rats also underwent a stair climbing activity test at 24 hours after injection where they were scored from 0 to 5 based on how many steps they climb (0=rat climbed no steps; 1=rat climbed one step; 2=rat climbed two steps; 3=rat climbed three steps; 4=rat climbed four steps; 5=rat climbed five steps). The rats in treated with non-capsulated B. coagulans (Group C1) and diclofenac sodium (Group E) climbed significantly more steps than the carrageenan control group (Group B) (FIG. 15D). The rats also underwent a motility test at 24 hours after injection where they were scored from 0 to 3 based on how easily the rat walked (0=rat did not walk, 1=rat walked with difficulty and avoided touching the toes of the inflamed paw to the floor; 2=rat walked with little difficulty, but with inflamed toe touching the floor, 3=rat walked easily). The rats treated with non-capsulated B. coagulans (Group C1) and diclofenac sodium (Group E) scored significantly higher in the motility test than the rats carrageenan control group (FIG. 15E). See Solanki, H. K. et al., “Evaluation of anti-inflammatory activity of probiotic on carrageenan-induced paw edema in Wistar rats,” International Journal of Biological Macromolecules 72 (2015) 1277-1282, herein incorporated by reference in its entirety.

### Example 14: Effect of Lactococcus lactis on Immune Response

A randomized, double-blind, placebo-controlled study investigated the effects of heat-killed Lactococcus lactis JCM5805 on pathogenesis of common cold and influenza. Subjects were divided into placebo (n=205) and treated (n=191) groups. Subjects consumed placebo capsules or JCM5805 capsules daily for 12 weeks. The JCM5085 capsules contained approximately 100 billion heat-killed JCM5085. Blood and saliva samples were taken before and after the 12 week intake period. Peripheral blood mononuclear cells (PMBCs) were isolated from the whole blood samples and were analyzed for HLA-DR levels and transcription levels of 18S RRNA, IFNA, VIPERIN, and IFITM1. The treated group experienced a significantly higher expression of HLA-DR on plasmytoid dendritic cells (pDC) (p<0.05) (FIG. 14A) and of VIPERIN in peripheral blood mononuclear cells (PMBCs) (p<0.001) after the treatment period when compared their baseline (FIG. 14B). The treated group also saw higher IFNA expression compared to the placebo group after the treatment period (p<0.05) (FIG. 14B). The increased pDC activation and increased anti-viral gene expression suggests L. lactis stimulates immune response to the common cold and influenza. See Shibata, T., et al., “Lactococcus lactis JCM5805 activates anti-viral immunity and reduces symptoms of common cold and influenza in healthy adults in a randomized controlled trial,” Journal of Functional Foods, 24 (2016) 492-500, herein incorporated by reference in its entirety.

A randomized, double-blind, placebo-controlled study investigated the effect of L. lactis on pathogenesis of influenza-like illness. Subjects were divided into a placebo group (n=107) and a treated group (n=106). The placebo group consumed a placebo beverage daily for 10 weeks; the treated group consumed a yogurt made with L. lactic JCM5805 daily for 10 weeks. Blood samples were collected from the subjects before and after the intake period. The treated group had higher IFN-α elicited by A/H1N1 (p=0.14, FIG. 15A) and higher ISG15 elicited by A/H1N1 (p<0.05, FIG. 13B) when compared to the placebo group. The increased IFN-α expression suggests that L. lactis is able to prevent the pathogenesis of influenza-like illness. See Sugimura, T. et al., “Effects of oral intake of plasmacytoid dendritic cells-stimulative lactic acid bacterial strain on pathogenesis of influenza-like illness and immunological response to influenza virus,” British Journal of Nutrition (2015), 114, 727-733, herein incorporated by reference in its entirety.

### Example 15: Effect of Lactococcus lactis on Influenza Incidence

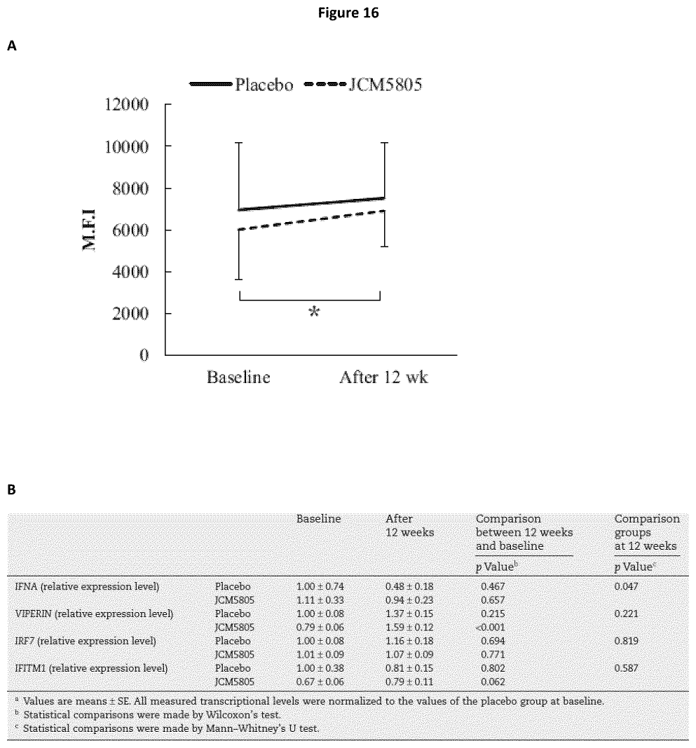

A community-based intervention studied the effect of L. lactis on influenza incidence rates in schoolchildren. The elementary schoolchildren and junior high school students in one town were given JCM-5085 yogurt at their schools. One serving of yogurt contained 100 billion cells of L. lactis. The incidence rates of influenza in the treated schools were compared to incidence rates at a nearby untreated school. The elementary schoolchildren and who consumed the JCM-5085 yogurt experienced a statistically significant reduction in cumulative incidence rates in influenza (p<0.01) (FIG. 16). The combined incidence rate of the elementary and junior high school students was also significantly decreased (p<0.01) (FIG. 16). See Sakata, K. et al., “Preventive Effect of Lactococcus lactis subsp. lactis JCM 5805 Yogurt Intake on Influenza Infection among Schoolchildren,” Health, 2017, 9, 756-762, herein incorporated by reference in its entirety.

### Example 16: Effect of Lactococcus lactis on Cold and Flu Symptoms

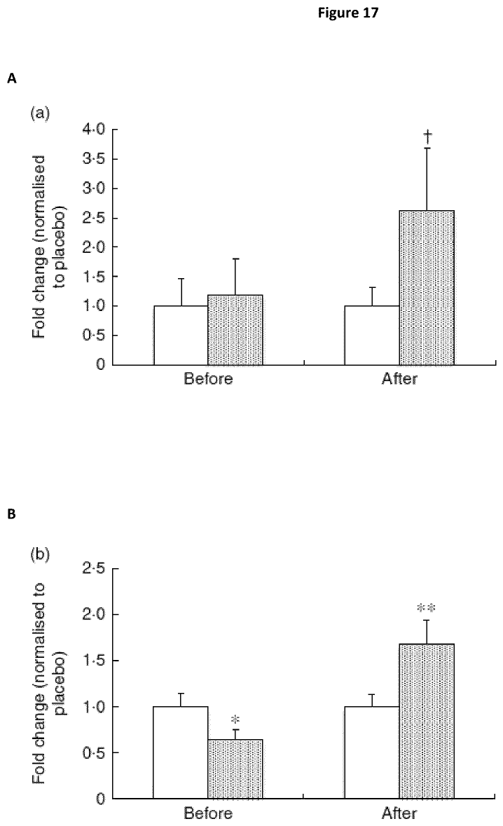

A randomized, double-blind, placebo-controlled study investigated the effects of heat-killed Lactococcus lactis JCM5805 on pathogenesis of common cold and influenza. Subjects were divided into placebo (n=205) and treated (n=191) groups. Subjects consumed placebo capsules or JCM5805 capsules daily for 12 weeks. The JCM5085 capsules contained approximately 100 billion heat-killed JCM5085. Subjects self-reported their symptoms daily. The treated group saw a significantly fewer number of incidence days of sore throat (p<0.05) and cough (p<0.001) (FIG. 17). This study suggests that L. lactis can reduce cold and flu symptoms. See Shibata, T., et al., “Lactococcus lactis JCM5805 activates anti-viral immunity and reduces symptoms of common cold and influenza in healthy adults in a randomized controlled trial,” Journal of Functional Foods, 24 (2016) 492-500, herein incorporated by reference in its entirety.

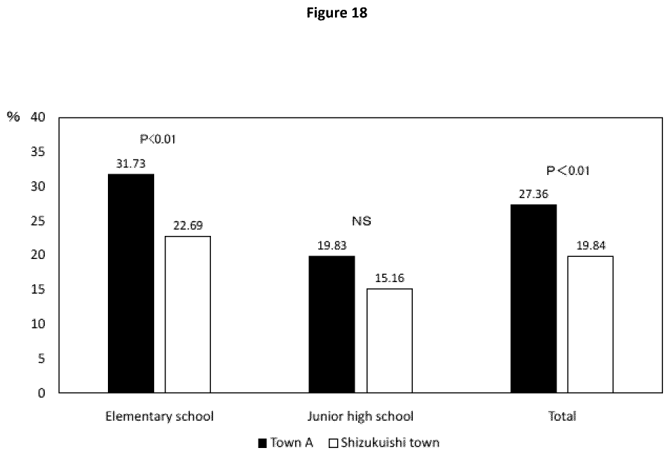

A randomized, double-blind, placebo-controlled study investigated the effect of L. lactis on pathogenesis of influenza-like illness. Subjects were divided into a placebo group (n=107) and a treated group (n=106). The placebo group consumed a placebo beverage daily for 10 weeks; the treated group consumed a yogurt made with L. lactic JCM5805 daily for 10 weeks. One serving of yogurt contained 1×10¹¹ CFUs of L. lactis. Subjects recorded daily their body temperature and symptom questionnaire. Incidence of cold and influenza were diagnosed by a medical doctor. The treated group experienced a reduction in cumulative days of incidence of cough (p<0.001) and feverishness (p<0.001) (FIG. 18A). The treated group also saw significant reduction in severe or moderate symptoms of cough (p=0.015), sore throat (p=0.009) and feverishness (p=0.009) (FIG. 18B). This study suggests that consumption of L. lactis can decrease incidence and severity of cold and flu symptoms. See Sugimura, T. et al., “Effects of oral intake of plasmacytoid dendritic cells-stimulative lactic acid bacterial strain on pathogenesis of influenza-like illness and immunological response to influenza virus,” British Journal of Nutrition (2015), 114, 727-733, herein incorporated by reference in its entirety.

### Example 17: Effect of Lactococcus lactis on Infectious Disease

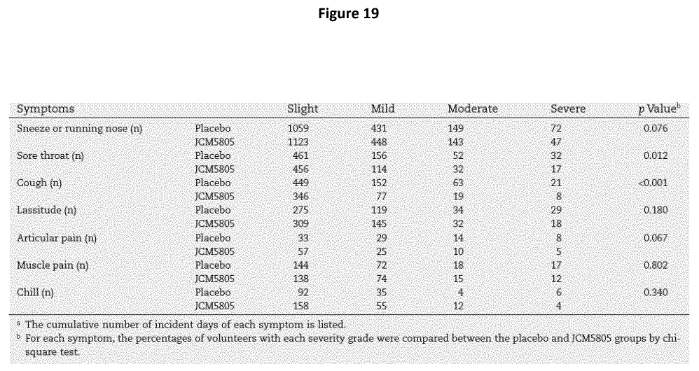

A randomized, placebo-controlled, double-blind, parallel group study investigated efficacy of L. lactis in reducing upper respiratory disease (URID) and gastrointestinal disease (GID) symptoms. Subjects were elementary schoolchildren divided into a placebo group (n=456) and a treated group (n=436). The treated group consumed a beverage containing L. lactis at a dose of 1×10¹¹ count of L. lactis. Subjects recorded their health condition in a record booklet. Each guardian of a subject also recorded the subject's body temperature and scores for each symptom. The treated group saw significant fewer days of absence from both URID and GID (p=0.004) and URID (p=0.005) (FIG. 19A). The students who consumed the treated beverage saw significantly fewer days of symptoms of fever (p=0.001), runny nose (p=0.07), constipation (p=0.1), diarrhea (p=0.01), and abdominal pain (p=0.1) (FIG. 19B). The students in the treated group also experienced higher cumulative days of general well-being (p<0.05) (FIG. 19C). See Nguyet Thu, N., et al., “Impact of Infectious Disease after Lactococcus lactis Strain Plasma Intake in Vietnamese Schoolchildren: A Randomized, Placebo-Controlled, Double-Blind Study,” Nutrients, 2022, 14, 552, herein incorporated by reference in its entirety.

### Example 18: Effect of Lactococcus lactis on Exercise Fatigue, Immunity, and Cold and Flu Symptoms

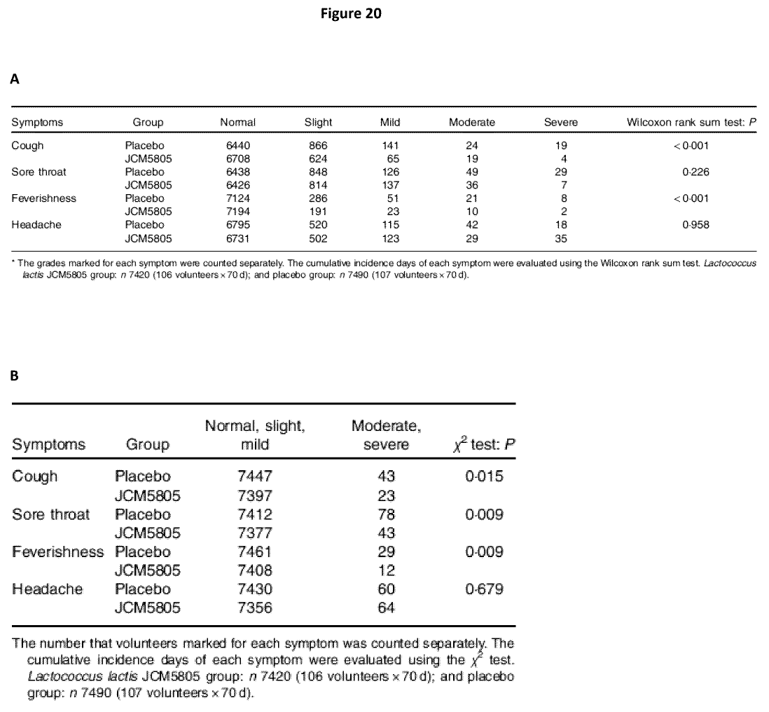

A randomized, placebo-controlled, double-blind study investigated the effect of L. lactis on fatigue and upper respiratory tract infections. Subjects belonged to a university sports club and were divided into a placebo group (n=25) and a treated group (n=26). The subjects performed high intensity exercise according to their club's training regimen throughout the intervention period. The placebo group consumed capsules containing cornstarch; the treated group consumed capsules containing approximately 100 billion cells of L. lactis. Blood and saliva samples were taken twice, before and after the intervention period. Subjects also reported their physical condition and symptoms daily. The treated group experienced a significant increase in CD86 compared to the placebo group (p<0.016, FIG. 20A) suggesting that L. lactis increases pDC maturation. The treated group also experienced significantly fewer cumulative days of incidence of upper respiratory tract infections (p=0.028, FIG. 20B) and cumulative days of sneeze or runny nose symptoms compared to the placebo group (p=0.032, FIG. 20C). The treated group experiences fewer days of poor physical condition (p=0.030), fatigue (p=0.032), and articular pain (p=0.016) (FIG. 20D). See Komano, Y. et al., “Efficacy of heat-killed Lactococcus lactis JCM 5805 on immunity and fatigue during consecutive high intensity exercise in male athletes: a randomized, placebo-controlled, double-blinded trial,” Journal of the International Society of Sports Nutrition, (2018) 15:39, herein incorporated by reference in its entirety.

### Example 19: Effect of Lactococcus lactis on Presenteeism

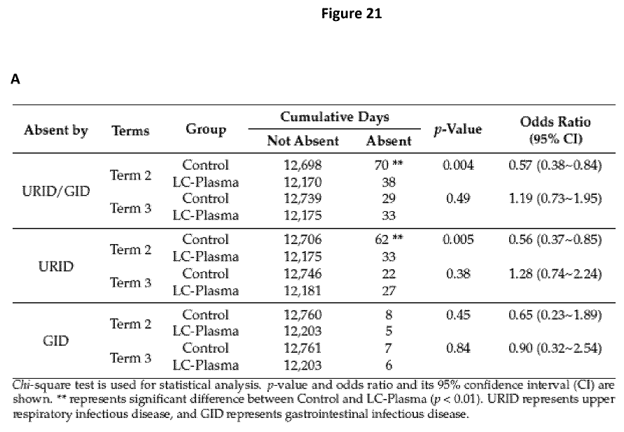

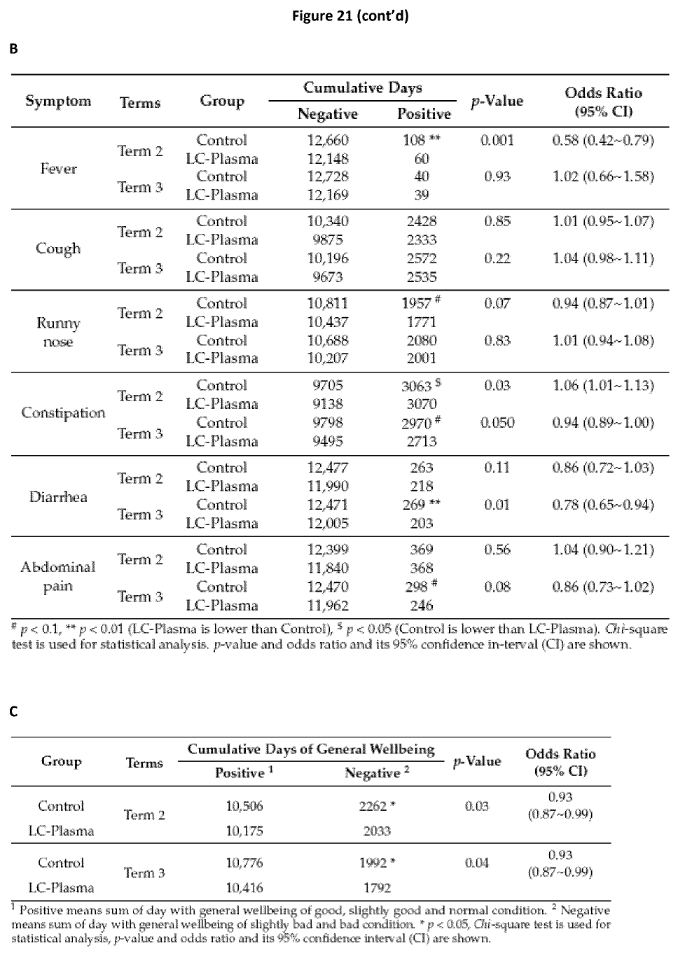

A randomized, open-label, crossover study investigated the effect of L. lactis on performance and physical condition of office workers. Subjects were divided into two groups. One group had an intake period followed by a non-intake period; the other group had a non-intake period followed by an intake period. The subjects were assessed using the World Health Organization Health and Work Performance Questionnaire and the Profile of Mood States questionnaire and physical condition questionnaire. The subjects' absolute presenteeism (p=0.04, FIG. 21A) and vigor (p 0.02, FIG. 21B) were superior during the intake period. The subjects also experienced fewer cumulative days of sneeze and runny nose (p=0.04), cough or sore throat (p<0.01), and lassitude (p=0.01) (FIG. 21C). Subject also reported improved physical condition during the intake period (p<0.01, FIG. 21C). These results suggest that L. lactis improves work performance and physical condition of employees. See Kokubo, T. et al., “Lactococcus lactis Strain Plasma Improves Subjective Physical State and Presenteeism: A Randomized, Open-Label Crossover Study among Healthy Office Workers,” Prev. Nutr. Food Sci. 2020; 25(2):140-145, herein incorporated by reference in its entirety.

### Example 20: Effect of Lactococcus lactis on Dengue Fever-Like Symptoms

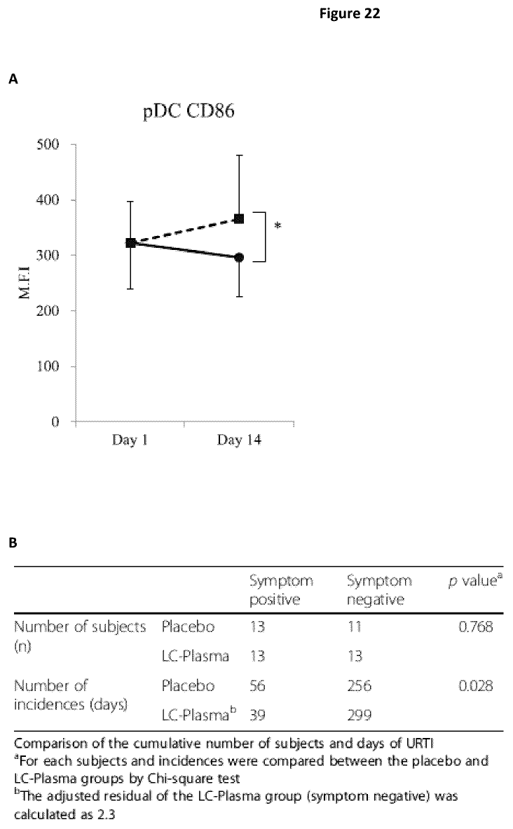

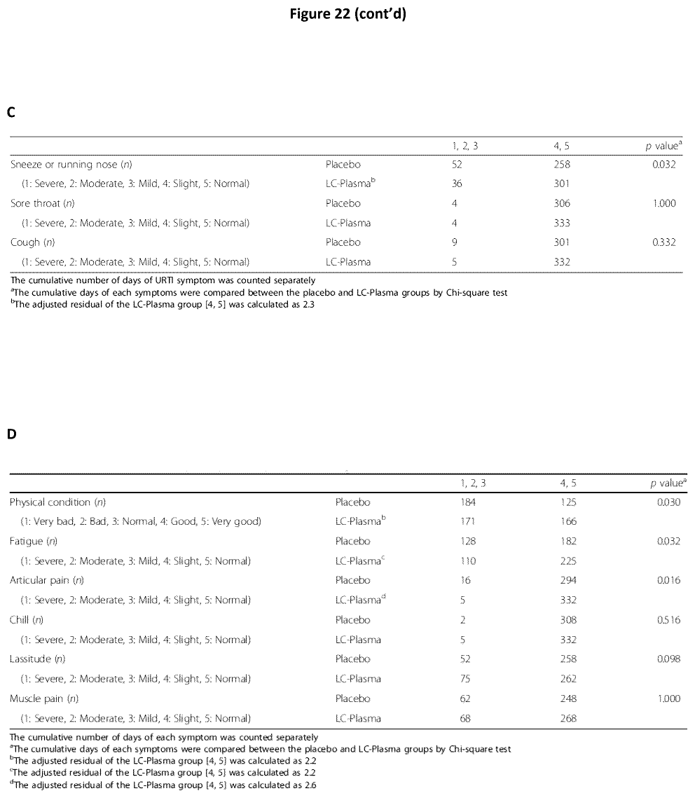

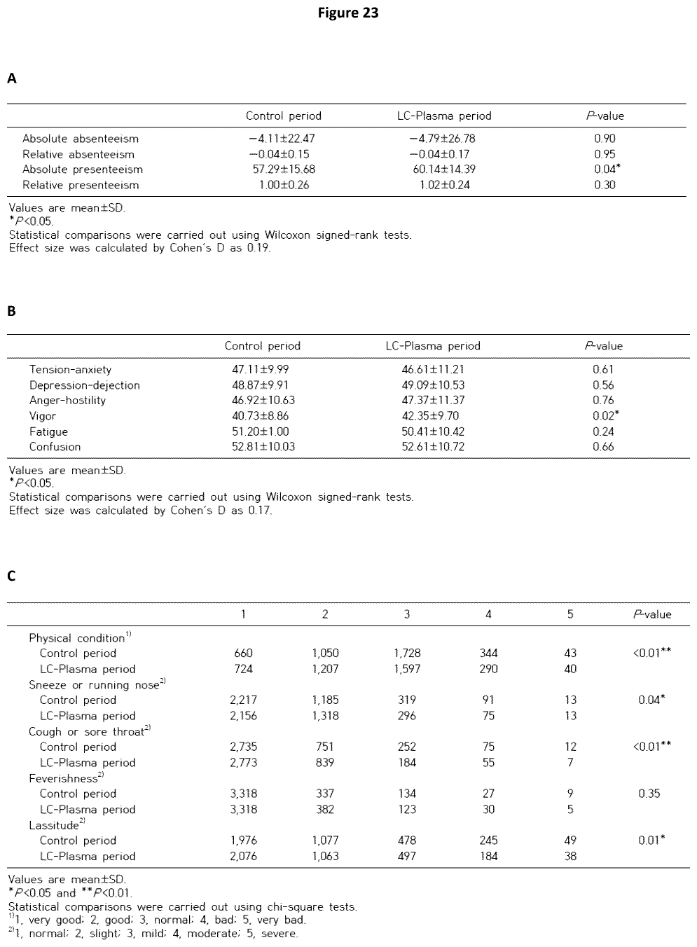

A randomized, placebo-controlled, double-blind, parallel-group trial studied the effect of L. lactis on dengue fever-like symptoms. Subjects were assigned to either the placebo group (n=53) or treatment group (n=50). The treatment group consumed tablets containing approximately 100 billion cells daily for 8 weeks. Participants recorded their symptoms through self-recorded questionnaires and were tested for exposure to the dengue virus. Subject who consumed L. lactis experienced a significant reduction in cumulative incidence days of dengue fever-like symptoms of fever (p<0.001), muscle pain (p<0.005), joint pain (p<0.001), pain behind the eyes (p<0.001), headache (P<0.001), sore throat (p<0.001), cough (p<0.05), runny nose (p<0.001), and sneezing (p<0.01) (FIG. 22). See Khor, C.-S., et al., “Lactococcus lactis Strain Plasma Intake Suppresses the Incidence of Dengue Fever-like Symptoms in Healthy Malaysians: A Randomized, Double-Blind, Placebo-Controlled Trial,” Nutrients 2021, 13, 4507, herein incorporated by reference in its entirety.

### Example 21: Effect of Lactococcus lactis on Skin Condition

A randomized, double-blind, placebo-controlled, parallel group study investigated the effect of L. lactis on skin immunity. Subjects were assigned to the placebo group (n=35) or the treatment group (n=35). Subjects in the treatment group were administered a capsule containing 100 billion cells of L. lactis daily for 8 weeks. The skin microbiome, gene expression, and skin conditions were monitored. Swabs from skin above the eyebrow were taken to analyze the skin microbiome. Hair follicles were isolated to observe gene expression. The group consuming L. lactis did not have a decrease in the ratio of Staphylococcus epidermidis (q<0.05, FIG. 23A), Staphylococcus pasteuri (q<0.05, FIG. 23B), and Sphingomonus roseiflava (q<0.1, FIG. 23C) on the skin when the placebo group did experience a decrease. The treated group also did not experience an increase in P. acnes (p<0.05, FIG. 23D) like the placebo group experienced. These results suggest that L. lactis helps maintain skin microbiome. The treated group also saw significant increases in expression levels of skin homeostasis genes TGFB1 (p=0.019), BD1 (p=0.02), BD3 (p=0.008), S100A9 (p=0.007), Cldn1 (p<0.001), and OCLN (p=0.026) (FIG. 23E). The treated group also saw a decrease in erythema (P=0.041) and did not see the increase in skin dryness experienced by the placebo group (p=0.003) (FIG. 23F). See Tsuji, R. et al., “The Effects of Dietary Supplementation of Lactococcus lactis Strain Plasma on Skin Microbiome and Skin Conditions in Healthy Subjects-A Randomized, Double-Blind, Placebo-Controlled Trial,” Microorganisms, 2021, 9, 563, herein incorporated by reference in its entirety.

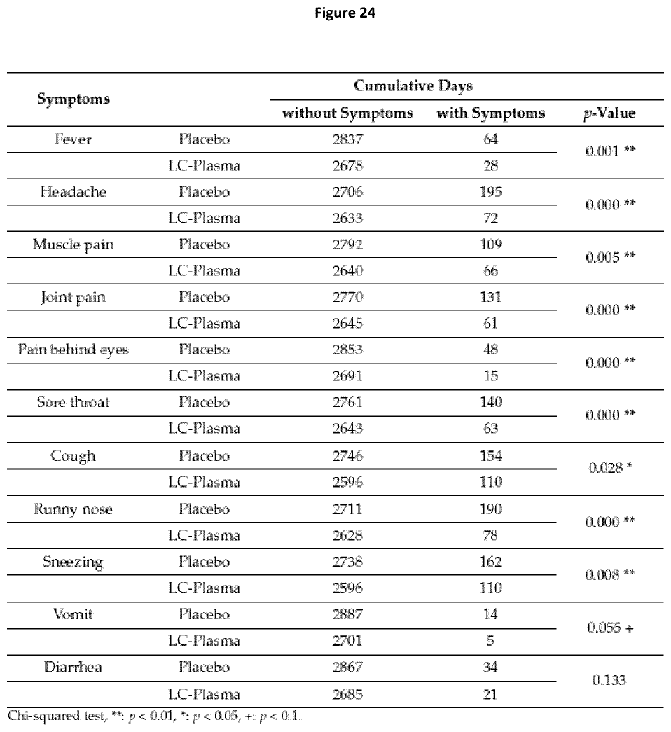

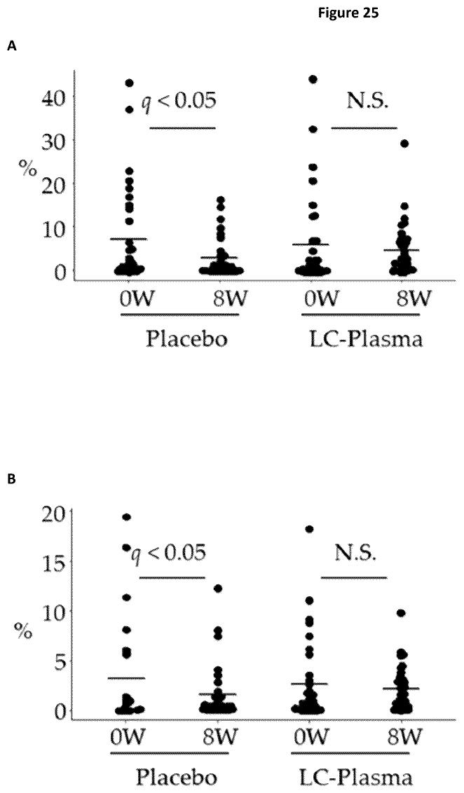

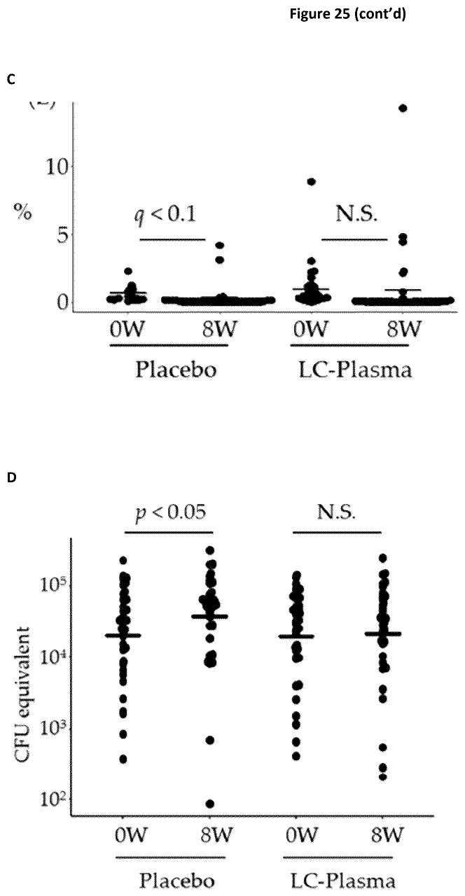

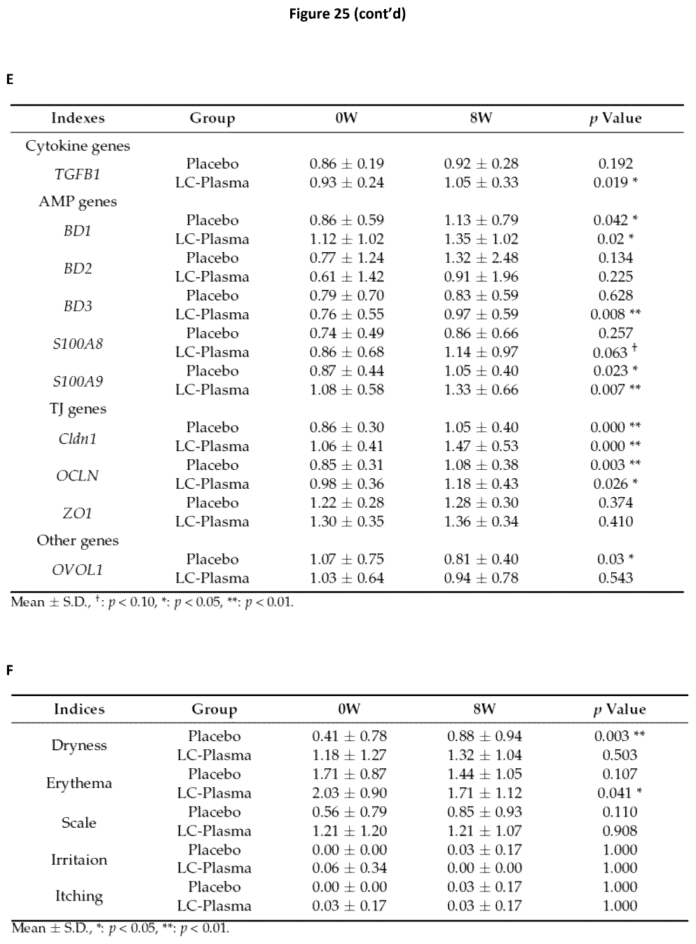

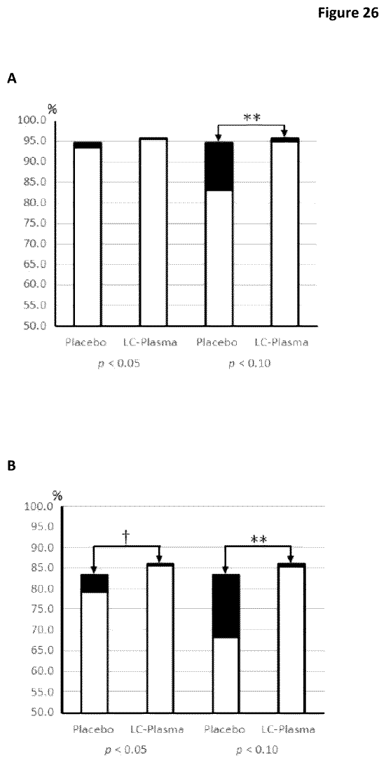

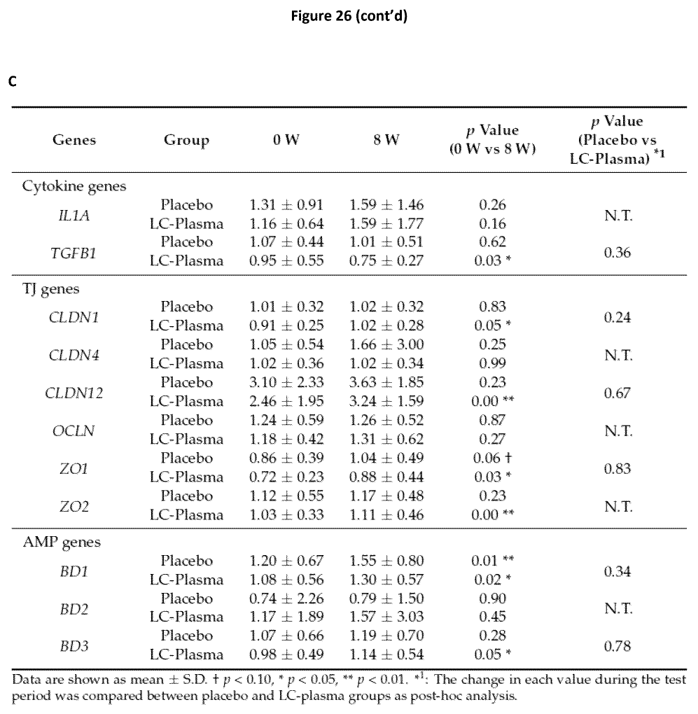

A randomized, double-blind, placebo-controlled study investigated the effect of L. lactis on skin microbiome and skin homeostasis-related genes. Subjects were divided into a placebo group (n=36) and a treatment group (n=35). The treated group consumed a capsule with 1×10¹¹ cells of L. lactis. Hair roots were obtained to analyze expression of skin-homeostasis genes. Cheek swabs were taken to analyze skin microbiota. Samples were taken before and after the intake period. The placebo group experienced significant changes in the genus and species of microbiota on the skin while the treated group did not experience significant changes (p<0.01, FIGS. 24A and 24B). The treated group experienced higher expression of skin-homeostasis genes CLDN1 (p=0.05), CLDN12 (p <0.01), ZO1 (p=0.03), ZO2 (p<0.01), BS1 (p=0.02), and BD3 (p=0.05), while TGFB1 was significantly down-regulated (p=0.03) (FIG. 24C). See Fujii, T. et al., “Effects of Heat-Killed Lactococcus lactis Strain Plasma on Skin Homeostasis-Related Genes and the Skin Microbiome among Healthy Adults: A Randomized Controlled Double-Blind Study,” Microorganisms 2021, 9, 2029, herein incorporated by reference in its entirety.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. Unless specifically set forth herein, the terms “a” “an” and “the” are not limited to one element but instead should be read as meaning “at least one”.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

1. An orally ingestible composition comprising:
an amount of gold kiwifruit powder,
an amount of Bacillus coagulans, and
an amount of heat-treated Lactococcus lactis.

2. The composition according to claim 1, wherein the gold kiwifruit powder comprises Livaux®.

3. The composition according to claim 1, wherein the Bacillus coagulans comprises Unique IS-2®.

4. The composition according to claim 1, wherein the Lactococcus lactis comprises IMMUSE™.

5. The composition according to claim 1, wherein the ratio of the amount of gold kiwifruit powder:the amount of Bacillus coagulans is between 2:1 and 15:1.

6. The composition according to claim 1, wherein the ratio of the amount of gold kiwifruit powder:the amount of Lactococcus lactis is between 5:1 and 20:1.

7. The composition according to claim 1, wherein the ratio of the amount of Bacillus coagulans:the amount of Lactococcus lactis is between 1:1 and 5:1.

8. The composition according to claim 1, wherein the orally ingestible composition is in the form of a gummy, a tablet, a capsule, a liquid, a suspension, a chewable, a soft gel, a sachet, a powder, a syrup, a liquid suspension, an emulsion, or solution.

9. The composition according to claim 1, wherein the orally ingestible composition is in the form of an individual oral dosage form.

10. The composition according to claim 1, wherein the individual oral dosage form is a gummy.

11. The composition according to claim 9, wherein the oral dosage form comprises between 75 mg and 700 mg gold kiwifruit powder; between 5 mg and 80 mg heat-treated Lactococcus lactis; and between 5 mg and 140 mg Bacillus coagulans.

12. The composition according to claim 9, wherein the individual oral dosage form comprises between about 500 million CFUs and 14 billion CFUs Bacillus coagulans.

13. The composition according to claim 9, wherein the oral dosage form comprises about 125 mg gold kiwifruit powder; about 18 mg Lactococcus lactis; and about 25 mg Bacillus coagulans.

14. The composition according to claim 9, wherein the oral dosage form comprises about 2.5 billion CFUs Bacillus coagulans.

15. A method of increasing the amount of Faecalibacterium prausnitzii within a digestive tract of a human subject, improving digestive health of a human subject, improving one or more GI symptoms in a human subject suffering from small intestinal bacterial overgrowth, alleviating IBS symptoms in a human subject suffering from IBS, or reducing constipation or relieving symptoms of constipation in a human subject, wherein the method comprises orally administering, to the human subject, an orally ingestible composition according to claim 1.

16.-28. (canceled)

29. A method of reducing inflammation caused by plaque-induced gingivitis in a human subject or improving oral health and reducing tooth decay in a human subject, wherein the method comprises orally administering, to the human subject, an orally ingestible composition according to claim 1.

30. (canceled)

31. A method of reducing bacterial vaginosis symptoms in a human female subject or reducing incidence of hepatic encephalopathy in a human subject suffering from liver cirrhosis, wherein the method comprises orally administering, to the human subject or human female subject, an orally ingestible composition according to claim 1.

32. (canceled)

33. A method of improving protein and amino acid absorption in a human subject or improving exercise or work performance, wherein the method comprises orally administering, to the human subject, an orally ingestible composition according to claim 1.

34.-38. (canceled)

39. A method of improving cholesterol levels of a human subject, maintaining or improving skin condition in a human subject, or reducing swelling in a human subject, wherein the method comprises orally administering, to the human subject, an orally ingestible composition according to claim 1.

40.-54. (canceled)

55. A method of improving immune health of a human subject, improving immune response of a human subject, or improving or alleviating one or more symptoms of an illness in a human subject, wherein the method comprises orally administering, to the human subject, an orally ingestible composition according to claim 1.

56.-80. (canceled)
